(19) United States
(12) Patent Application Publication
(10) Pub. No.: US 2025/0002921 A1
(43) Pub. Date: Jan. 2, 2025

(54) SPHERICAL NUCLEIC ACIDS FOR cGAS-STING AND STAT3 PATHWAY MODULATION FOR THE IMMUNOTHERAPEUTIC TREATMENT OF CANCER

(71) Applicant: NORTHWESTER UNIVERSITY, Evanston, IL (US)

(72) Inventors: Alexander H. Stegh, Chicago, IL (US); Chad A. Mirkin, Wilmette, IL (US); Akanksha Sanjay Mahajan, Evanston, IL (US); Michelle Hope Teplensky, Evanston, IL (US); Jungsoo Park, Evanston, IL (US); Matthew Kuo Vasher, Pickney, MI (US); Andrew Joseph Sinegra, Chicago, IL (US)

(21) Appl. No.: 18/710,942

(22) PCT Filed: Nov. 17, 2022
(86) PCT No.: PCT/US22/80086

§ 371 Date: May 16, 2024

Related U.S. Application Data
(60) Provisional application No. 63/280,499, filed on Nov. 17, 2021.

(51) Int. Cl. C12N 15/117; A61P 35/00

(52) U.S. Cl. CPC C12N 15/117; A61P 35/00; C12N 2310/17; C12N 2310/351

(57) ABSTRACT

The disclosure is generally directed to spherical nucleic acids (SNAs), nanostructures with a core surrounded by a radial presentation of oligonucleotides, that can activate a cytoplasmic DNA sensor including but not limited to cyclic GMP-AMP synthase (cGAS). In some embodiments, the SNAs also inactivate a transcription factor including but not limited to signal transducer and activator of transcription 3 (STATS). Methods of making and using the SNAs are also provided herein. In some aspects, the present disclosure provides a spherical nucleic acid (SNA) comprising (a) a nanoparticle core; and (b) a shell of oligonucleotides attached to the external surface of the nanoparticle core, the shell of oligonucleotides comprising a double-stranded or single-stranded stem loop DNA oligonucleotide that activates cyclic GMP-AMP synthase (cGAS) and is at least 15 base pairs in length.

### CROSS-REFERENCE TO RELATED APPLICATIONS

This application claims the priority benefit under 35 U.S.C. § 119(e) of U.S. Provisional Patent Application No. 63/280,499, filed Nov. 17, 2021, which is incorporated herein by reference in its entirety.

### STATEMENT OF GOVERNMENT INTEREST

This invention was made with government support under grant numbers 1R01CA208783-01A1 and 5U54CA199091-05 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-221_sequence_listing.xml”, which was created on Nov. 17, 2022 and is 9,693 bytes in size. The subject matter of the Sequence Listing is incorporated herein in its entirety by reference.

### FIELD

The disclosure is generally related to spherical nucleic acids (SNAs), nanostructures with a core surrounded by a radial presentation of oligonucleotides, that can activate cyclic GMP-AMP synthase (cGAS). In some embodiments, the SNAs also inactivate signal transducer and activator of transcription 3 (STAT3). Methods of making and using the SNAs are also provided herein.

### BACKGROUND

Activation of the Stimulator of Interferon Genes (STING) pathway represents one of the main immune sensing mechanisms that promotes innate and adaptive immune responses against tumor¹. Tumor-derived DNA is recognized by cyclic GMP-AMP synthase (cGAS) in antigen-presenting cells. Upon nucleic acid recognition, cGAS generates cyclic dinucleotide GMP-AMP (cGAMP). cGAMP in turn binds to and activates the adaptor protein STING and triggers Interferon Regulatory Factor 3 (IRF3) and Nuclear Factor-κB (NF-κB)-dependent transcription, to promote the activation of natural killer (NK) cells, pro-inflammatory macrophages, and T cells¹.

### SUMMARY

Intratumoral administration of STING-agonistic cyclic dinucleotides (CDNs) antagonizes tumor progression in multiple cancer models, including orthotopic glioblastoma (GBM) models², and is currently being tested in a phase I clinical trial in patients with advanced non-CNS cancers. Limited stability and bioavailability together with insufficient lipophilicity, however, limit clinical CDN development, in particular when consideration is given to non-invasive treatment approaches.

Spherical Nucleic Acids (SNAs) are modular structures comprising a nanoparticle core densely functionalized with a shell of radially oriented oligonucleotides³. The unique three-dimensional architecture confers enhanced resistance to nuclease-mediated degradation and allows for robust cell entry and bioavailability without requiring auxiliary delivery vehicles. Early phase clinical trials of siRNA-based SNAs (SNA^{siRNA}) in glioblastoma (GBM)⁴ and toll-like receptor (TLR) 9-agonistic SNAs carrying CpG-rich DNA oligonucleotides (SNAs^{CpG}) in solid cancers^{5,6} provided the first clinical evidence that SNAs represent a safe, brain-penetrant therapy for gene regulation and immunostimulation in GBM and other solid tumors.

Advantages of the technology provided herein include, but are not limited to:
SNAs conjugated with Interferon-stimulatory (ISD) oligonucleotides (SNAs^{ISD}) to directly activate cGAS outperform CDNs targeted to STING currently under clinical development, in particular, when consideration is given to non-invasive delivery approaches.
Bimodal SNAs designed to engage both cGAS and STAT3 protein (SNAs^{ISD/STAT3i}) concomitantly target orthogonal signaling pathways, to achieve more effective anti-tumor immunity.

Accordingly, in some aspects the disclosure provides a spherical nucleic acid (SNA) comprising (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that activates cyclic GMP-AMP synthase (cGAS) and is at least 15 base pairs in length. In some aspects, the disclosure provides a spherical nucleic acid (SNA) comprising (a) a nanoparticle core; and (b) a shell of oligonucleotides attached to the external surface of the nanoparticle core, the shell of oligonucleotides comprising a double-stranded DNA oligonucleotide that activates cyclic GMP-AMP synthase (cGAS) and is at least 15 base pairs in length. In some aspects, the disclosure provides a spherical nucleic acid (SNA) comprising (a) a nanoparticle core; and (b) a shell of oligonucleotides attached to the external surface of the nanoparticle core, the shell of oligonucleotides comprising a single-stranded stem loop DNA oligonucleotide that activates cyclic GMP-AMP synthase (cGAS) and is at least 15 base pairs in length. In some embodiments, the shell of oligonucleotides comprises a plurality of double-stranded DNA oligonucleotides and/or single-stranded stem loop DNA oligonucleotides, each of which activates cGAS and is at least 15 base pairs in length. In some embodiments, the shell of oligonucleotides comprises a plurality of double-stranded DNA oligonucleotides, single-stranded stem loop DNA oligonucleotides, or a combination thereof, each of which activates cGAS and is at least 15 base pairs in length. In some embodiments, the shell of oligonucleotides consists of a plurality of double-stranded DNA oligonucleotides and/or single-stranded stem loop DNA oligonucleotides, each of which activates cGAS and is at least 15 base pairs in length. In some embodiments, the shell of oligonucleotides consists of a plurality of double-stranded DNA oligonucleotides, single-stranded stem loop DNA oligonucleotides, or a combination thereof, each of which activates cGAS and is at least 15 base pairs in length. In some embodiments, the shell of oligonucleotides comprises a plurality of double-stranded DNA oligonucleotides and each of the plurality of double-stranded DNA oligonucleotides comprises one strand comprising SEQ ID NO: 3 and another strand comprising SEQ ID NO: 4. In some embodiments, the shell of oligonucleotides consists of a plurality of double-stranded DNA oligonucleotides and each of the plurality of double-stranded DNA oligonucleotides comprises one strand comprising SEQ ID NO: 3 and another strand comprising SEQ ID NO: 4. In some embodiments, the double-stranded DNA oligonucleotide and/or single-stranded stem loop DNA oligonucleotide inactivates signal transducer and activator of transcription 3 (STAT3). In some embodiments, the double-stranded DNA oligonucleotide inactivates signal transducer and activator of transcription 3 (STAT3). In some embodiments, the single-stranded stem loop DNA oligonucleotide inactivates signal transducer and activator of transcription 3 (STAT3). In some embodiments, the shell of oligonucleotides comprises a plurality of double-stranded DNA oligonucleotides and/or single-stranded stem loop DNA oligonucleotides, each of which activates cGAS, inactivates signal transducer and activator of transcription 3 (STAT3), and is at least 15 base pairs in length. In some embodiments, the shell of oligonucleotides comprises a plurality of double-stranded DNA oligonucleotides, each of which activates cGAS, inactivates signal transducer and activator of transcription 3 (STAT3), and is at least 15 base pairs in length. In some embodiments, the shell of oligonucleotides comprises a plurality of single-stranded stem loop DNA oligonucleotides, each of which activates cGAS, inactivates signal transducer and activator of transcription 3 (STAT3), and is at least 15 base pairs in length. In some embodiments, the shell of oligonucleotides consists of a plurality of double-stranded DNA oligonucleotides and/or single-stranded stem loop DNA oligonucleotides, each of which activates cGAS, inactivates signal transducer and activator of transcription 3 (STAT3), and is at least 15 base pairs in length. In further embodiments, the shell of oligonucleotides consists of a plurality of double-stranded DNA oligonucleotides, each of which activates cGAS, inactivates signal transducer and activator of transcription 3 (STAT3), and is at least 15 base pairs in length. In some embodiments, the shell of oligonucleotides consists of a plurality of single-stranded stem loop DNA oligonucleotides, each of which activates cGAS, inactivates signal transducer and activator of transcription 3 (STAT3), and is at least 15 base pairs in length. In various embodiments, the nanoparticle core is a metallic core, a semiconductor core, an insulator core, an upconverting core, a micellar core, a dendrimer core, a liposomal core, a polymer core, a metal-organic framework core, a lipid nanoparticle core, a protein core, or a combination thereof. In further embodiments, the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan. In various embodiments, the nanoparticle core comprises gold, silver, platinum, aluminum, palladium, copper, cobalt, indium, cadmium selenide, iron oxide, fullerene, metal-organic framework, silica, zinc sulfide, or nickel. In some embodiments, the lipid nanoparticle core comprises an ionizable lipid, a phospholipid, a sterol, and a lipid-polyethylene glycol (lipid-PEG) conjugate. In further embodiments, each oligonucleotide in the shell of oligonucleotides is covalently attached to the exterior of the lipid nanoparticle core through the lipid-PEG conjugate. In some embodiments, the liposomal core comprises a plurality of lipid groups. In further embodiments, the plurality of lipid groups comprises a lipid selected from the group consisting of the phosphatidylcholine, phosphatidylglycerol, and phosphatidylethanolamine families of lipids. In various embodiments, the lipid is selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE). In some embodiments, at least one oligonucleotide in the shell of oligonucleotides is attached to the exterior of the liposomal or lipid nanoparticle core through a lipid anchor group. In some embodiments, the lipid anchor group is attached to the 5′ end or the 3′ end of the at least one oligonucleotide. In further embodiments, the lipid anchor group is tocopherol, DOPE lipid, or cholesterol. In further embodiments, the shell of oligonucleotides comprises one or more additional oligonucleotides. In various embodiments, the one or more additional oligonucleotides comprises DNA, RNA, or a combination thereof. In further embodiments, the one or more additional oligonucleotides comprises single-stranded DNA, double-stranded DNA, single-stranded RNA, double-stranded RNA, or a combination thereof. In still further embodiments, the one or more additional oligonucleotides is an immunostimulatory oligonucleotide, an inhibitory oligonucleotide, an oligonucleotide that inactivates signal transducer and activator of transcription 3 (STAT3), or a combination thereof. In some embodiments, the inhibitory oligonucleotide is an antisense oligonucleotide, small interfering RNA (siRNA), an aptamer, a short hairpin RNA (shRNA), a DNAzyme, or an aptazyme. In some embodiments, the immunostimulatory oligonucleotide is a CpG-motif containing oligonucleotide, a double-stranded DNA oligonucleotide, or a single-stranded RNA oligonucleotide. In some embodiments, the immunostimulatory oligonucleotide is a toll-like receptor (TLR) agonist. In various embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In further embodiments, the inhibitory oligonucleotide is an antisense oligonucleotide, small interfering RNA (siRNA), an aptamer, a short hairpin RNA (shRNA), a DNAzyme, or an aptazyme. In some embodiments, a SNA of the disclosure further comprises an antigen. In some embodiments, the antigen is attached to one or more oligonucleotides in the shell of oligonucleotides. In some embodiments, the antigen is attached to the surface of the SNA. In some embodiments, the antigen is encapsulated in the nanoparticle core. In various embodiments, the antigen is a tumor associated antigen, a tumor specific antigen, a neo-antigen, or a combination thereof. In further embodiments, the antigen is OVA1, MSLN, P53, Ras, mutant IDH1 (IDH1 R132H), a melanoma related antigen, a HPV related antigen, a prostate cancer related antigen, a glioblastoma antigen, a grade IV astrocytoma antigen, an ovarian cancer related antigen, a breast cancer related antigen, a hepatocellular carcinoma related antigen, a bowel cancer related antigen, or human papillomavirus (HPV) E7 nuclear protein. In various embodiments, the SNA is from about 1 to about 150 nanometers (nm) in diameter. In further embodiments, the shell of oligonucleotides comprises about 4 to about 250 oligonucleotides. In still further embodiments, each oligonucleotide in the shell of oligonucleotides is about 15 to about 100 base pairs in length.

In some aspects, the disclosure provides a composition comprising a plurality of spherical nucleic acids (SNAs) of the disclosure.

In further aspects, the disclosure provides a method of producing an immune response to cancer in a subject, comprising administering to the subject an effective amount of a spherical nucleic acid (SNA) of the disclosure, a composition of the disclosure, or a combination thereof, thereby producing an immune response to cancer in the subject. In still further aspects, the disclosure provides a method of treating and/or ameliorating a cancer in a subject comprising administering to the subject an effective amount of a SNA of the disclosure, a composition of the disclosure, or a combination thereof. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the cancer is glioblastoma. In various embodiments, the administering is by oral administration, topical administration, intravenous administration, intraarterial administration, mucosal administration, intraperitoneal administration, intramuscular administration, intratumoral administration, parenteral administration, intradermal administration, intranasal administration, subcutaneous administration, or a combination thereof. In some embodiments, the administering is by direct intracranial/intratumoral administration. In some embodiments, the administering is combined with focused ultrasound (FUS), co-administration of microbubbles to temporarily open the blood-brain barrier, or a combination thereof. In some embodiments, the subject is female. In some embodiments, the subject is male. In some embodiments, the administering is by intranasal administration.

In further aspects, the disclosure provides a spherical nucleic acid (SNA) comprising: (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that activates a cytoplasmic DNA sensor and is at least 15 base pairs in length. In some embodiments, the shell of oligonucleotides comprises a plurality of double-stranded DNA oligonucleotides and/or single-stranded stem loop DNA oligonucleotides, each of which activates the cytoplasmic DNA sensor and is at least 15 base pairs in length. In some embodiments, the shell of oligonucleotides consists of a plurality of double-stranded DNA oligonucleotides and/or single-stranded stem loop DNA oligonucleotides, each of which activates the cytoplasmic DNA sensor and is at least 15 base pairs in length. In further embodiments, the double-stranded DNA oligonucleotide and/or single-stranded stem loop DNA oligonucleotide inactivates a transcription factor. In some embodiments, the shell of oligonucleotides comprises a plurality of double-stranded DNA oligonucleotides and/or single-stranded stem loop DNA oligonucleotides, each of which activates the cytoplasmic DNA sensor, inactivates the transcription factor, and is at least 15 base pairs in length. In some embodiments, the shell of oligonucleotides comprises or consists of one or more G-quartet oligonucleotides. In further embodiments, the shell of oligonucleotides consists of a plurality of double-stranded DNA oligonucleotides and/or single-stranded stem loop DNA oligonucleotides, each of which activates the cytoplasmic DNA sensor, inactivates the transcription factor, and is at least 15 base pairs in length. In various embodiments, the cytoplasmic DNA sensor is cyclic GMP-AMP synthase (cGAS), AIM2 (Absent in Melanoma-2), RNA polymerase Ill, DAI (DNA-dependent activator of IFN-regulatory factors), IFI16 (Interferon-γ-inducible protein 16), or a combination thereof. In any of the aspects or embodiments of the disclosure, the transcription factor promotes cancer progression. In various embodiments, the transcription factor is signal transducer and activator of transcription 3 (STAT3), cMyc, NANOG, SOX2, OCT4, or a combination thereof. In some embodiments, the shell of oligonucleotides comprises a plurality of double-stranded DNA oligonucleotides and each of the plurality of double-stranded DNA oligonucleotides comprises one strand comprising SEQ ID NO: 3 and another strand comprising SEQ ID NO: 4. In some embodiments, the shell of oligonucleotides consists of a plurality of double-stranded DNA oligonucleotides and each of the plurality of double-stranded DNA oligonucleotides comprises one strand comprising SEQ ID NO: 3 and another strand comprising SEQ ID NO: 4. In various embodiments, the nanoparticle core is a metallic core, a semiconductor core, an insulator core, an upconverting core, a micellar core, a dendrimer core, a liposomal core, a polymer core, a metal-organic framework core, a lipid nanoparticle core, a protein core, or a combination thereof. In various embodiments, the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan. In various embodiments, the nanoparticle core comprises gold, silver, platinum, aluminum, palladium, copper, cobalt, indium, cadmium selenide, iron oxide, fullerene, metal-organic framework, silica, zinc sulfide, or nickel. In some embodiments, the lipid nanoparticle core comprises an ionizable lipid, a phospholipid, a sterol, and a lipid-polyethylene glycol (lipid-PEG) conjugate. In some embodiments, each oligonucleotide in the shell of oligonucleotides is covalently attached to the exterior of the lipid nanoparticle core through the lipid-PEG conjugate. In some embodiments, the liposomal core comprises a plurality of lipid groups. In further embodiments, the plurality of lipid groups comprises a lipid selected from the group consisting of the phosphatidylcholine, phosphatidylglycerol, and phosphatidylethanolamine families of lipids. In still further embodiments, the lipid is selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE). In various embodiments, at least one oligonucleotide in the shell of oligonucleotides is attached to the exterior of the liposomal or lipid nanoparticle core through a lipid anchor group. In some embodiments, the lipid anchor group is attached to the 5′ end or the 3′ end of the at least one oligonucleotide. In some embodiments, the lipid anchor group is tocopherol, DOPE lipid, or cholesterol. In further embodiments, the shell of oligonucleotides comprises one or more additional oligonucleotides. In various embodiments, the one or more additional oligonucleotides comprises DNA, RNA, or a combination thereof. In further embodiments, the one or more additional oligonucleotides comprises single-stranded DNA, double-stranded DNA, single-stranded RNA, double-stranded RNA, or a combination thereof. In some embodiments, the one or more additional oligonucleotides is an immunostimulatory oligonucleotide, an inhibitory oligonucleotide, an oligonucleotide that inactivates signal transducer and activator of transcription 3 (STAT3), or a combination thereof. In some embodiments, the inhibitory oligonucleotide is an antisense oligonucleotide, small interfering RNA (siRNA), an aptamer, a short hairpin RNA (shRNA), a DNAzyme, or an aptazyme. In further embodiments, the immunostimulatory oligonucleotide is a CpG-motif containing oligonucleotide, a double-stranded DNA oligonucleotide, or a single-stranded RNA oligonucleotide. In some embodiments, the immunostimulatory oligonucleotide is a toll-like receptor (TLR) agonist. In various embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In various embodiments, the inhibitory oligonucleotide is an antisense oligonucleotide, small interfering RNA (siRNA), an aptamer, a short hairpin RNA (shRNA), a DNAzyme, or an aptazyme. In some embodiments, a SNA of the disclosure further comprises an antigen. In various embodiments, the antigen is attached to one or more oligonucleotides in the shell of oligonucleotides. In some embodiments, the antigen is attached to the surface of the SNA. In some embodiments, the antigen is encapsulated in the nanoparticle core. In various embodiments, the antigen is a tumor associated antigen, a tumor specific antigen, a neo-antigen, or a combination thereof. In further embodiments, the antigen is OVA1, MSLN, P53, Ras, mutant IDH1 (IDH1 R132H), mutant telomerase reverse transcriptase, a melanoma related antigen, a HPV related antigen, a prostate cancer related antigen, a glioblastoma antigen, a grade IV astrocytoma antigen, an ovarian cancer related antigen, a breast cancer related antigen, a hepatocellular carcinoma related antigen, a bowel cancer related antigen, or human papillomavirus (HPV) E7 nuclear protein. In various embodiments, the mutant telomerase reverse transcriptase is TERT; C228T, C250T, or a combination thereof. In some embodiments, the SNA is from about 1 to about 150 nanometers (nm) in diameter. In some embodiments, the shell of oligonucleotides comprises about 4 to about 250 oligonucleotides. In some embodiments, each oligonucleotide in the shell of oligonucleotides is about 15 to about 100 base pairs in length. In some aspects, the disclosure provides a composition comprising a plurality of the spherical nucleic acids (SNAs) of the disclosure. In some aspects, the disclosure provides a method of producing an immune response to cancer in a subject, comprising administering to the subject an effective amount of a spherical nucleic acid (SNA) of the disclosure, a composition of the disclosure, or a combination thereof, thereby producing an immune response to cancer in the subject. In some aspects, the disclosure provides a method of treating and/or ameliorating a cancer in a subject comprising administering to the subject an effective amount of a SNA of the disclosure, a composition of the disclosure, or a combination thereof. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the cancer is glioblastoma. In various embodiments, the administering is by direct intracranial/intratumoral administration, oral administration, topical administration, intravenous administration, intraarterial administration, mucosal administration, intraperitoneal administration, intramuscular administration, intratumoral administration, parenteral administration, intradermal administration, intranasal administration, subcutaneous administration, or a combination thereof. In some embodiments, the subject is female. In some embodiments, the subject is male. In some embodiments, the administering is by intranasal administration. In further embodiments, the administering is combined with focused ultrasound (FUS), co-administration of microbubbles to temporarily open the blood-brain barrier, or a combination thereof.

### BRIEF DESCRIPTION OF THE DRAWINGS

FIG. 1 shows that ISD45-SNAs directly engage cGAS. (A) Schematic of SNA architecture. (B) Cell-free cGAS activation assay. (C) Reporter assay to quantify IRF induction Median+/−standard deviation is shown. *p<0.05.

FIG. 2 shows that ISD45 SNAs induce IRF and pro-inflammatory cytokines. (A) ICP-MS to quantify cellular level of gold in cells treated with ISD45-SNAs. (B-D) Luciferase reporter assay to quantify IRF induction in RAW-Lucia™ macrophages (B), THP cells (C), and in WT, cGAS-, STING- and IRF-deficient RAW-Lucia™ macrophages. TFX, transfection agent. Median+/−standard deviation is shown. *p<0.05. (E) Multiplex ELISA-based and (F) antibody array-based cytokine profiling in RAW-Lucia™ macrophages.

FIG. 3 shows that cGAS agonism is mediated by the oligonucleotide shell, independent of core material and size (A) Schematic of PLGA-SNAs. (B) Uptake of PLGA^{Cy5}-based ISD45-SNAs in RAW Lucia™ macrophages pretreated with cytochalasin D or fucoidan. (C) Luciferase reporter assay in RAW Lucia™ macrophages. (D) Dose-dependent IRF3 activation in response to PLGA-based ISD45-SNAs and AduroS100.

FIG. 4 shows that ISD45-SNAs promote M1 macrophage polarization, tumor cell death and survival of GBM bearing mice. (A) Immunoblot of macrophage lysates showing abundance of the indicated cGAS-STING-pathway components. (B) Nitrite levels as determined in cell supernatant are indicated at the bottom. (C) Growth curves of CT2A tumor cells co-cultured with macrophages or exposed to supernatant from macrophages left untreated or treated with the ssDNA⁴⁵-SNAs or ISD45-SNAs. (D) MTT viability assays of CT2A tumor cells incubated with macrophage supernatants. Median+/−standard deviation is shown. (E) H&E staining and MRI scan of tumor-bearing mouse brain injected (via direct intratumoral administration) with Gd(III)-labeled SNAs. (F) Kaplan-Meyer survival curves of female BL6/C57 mice, inoculated with CT2A glioma tumor cells, and treated with ssDNA45- or ISD45-SNAs via direct intratumoral administration. *p<0.05.

FIG. 5 depicts nose-to-brain delivery of ISD45-SNAs. (A) Schematic of the nasal cavity and olfactory/trigeminal nerve pathways for drug delivery to the CNS. (B) gold ISD45-SNAs accumulate in brain/brain tumor parenchyma per ICP-MS analysis of gold in different organs, including lung, liver, kidney, spleen and the nasal/oral cavity. (C) Cy5-labeled gold-based ISD45-SNAs. (D-E) Fluorescence microcopy of tumor cells or a cross section of the trigeminal nerve shows accumulation of Cy5-labeled ISD45-SNAs in tumor and localized within the epineurium of the trigeminal nerve. (F) Schematic of Cy5-labeled PLGA-SNAs. Note, the Cy5 is attached to the core. (G) IVIS images of Head (including nasal cavity) and brain. (H) Fluorescence microscopy of cross sections indicate PLGA-SNACy5 accumulation in olfactory bulb and in different tumor sections. (I) IVIS and fluorescence images of isolated trigeminal nerves. Cross and longitudinal sections are shown. (A) Taken from Balyasnikova and colleagues, Expert Opinion Drug Delivery, 2018.

FIG. 6 shows that anti-tumor effect of intranasally delivered ISD45-SNAs is dependent on STING and can be augmented by checkpoint inhibition. (A-B) Quantification of bioluminescence in CT2A-bearing C57BL/6-wt mice (A) and in STING^{gt/gt} mice (B). (C) Quantification of bioluminescence of CT2A-bearing C57BL/6-wt mice left untreated, or treated with either ssDNA45-SNAs, ISD45-SNAs or the CDN AduroS100. Mean+/−standard deviations is shown. (D-G) Kaplan Meyer survival analyses in CT2A-bearing C57BL/6-wt mice (F), STING^{gt/gt} mice (E), and in CT2A-bearing C57BL/6-wt mice co-treated with SNAs and checkpoint inhibitors (G). In panel D, overall survival and survival in female and male animal subjects is shown. In panel E, median survival is indicated in days.

FIG. 7 shows ISD45-SNA anti-tumor effect can be augmented by co-treatment with checkpoint inhibitors (CPIs). (A) Kaplan-Meyer survival curves (overall, female and male animal subjects) of CT2A tumor-bearing mice. (B) Long-term survivor (female, identified in experiments in panel A) were rechallenged with CT2A tumor cells implanted into the contralateral hemisphere. Survival was analyzed using the Kaplan-Meyer method.

FIG. 8 depicts immune profiles upon nose-to-brain delivery of ISD45-SNAs. (A-B) Heat maps comparing effector T cell (A) and TAM content (B) in tumors isolated from mice left untreated or intranasally treated with ssDNA45- and ISD45-SNAs. (C—F) Minimum weight spanning trees (C) and histograms (D-F) showing enrichment of NK cell populations in deep cervical draining lymph nodes from mice treated with ISD45-SNAs. Mean+/−standard deviations is shown. * p<0.05.

FIG. 9 shows Bimodal SNAs^{ISD/STAT3i}. (A) Schematics of SNAs architectures designed for bimodal cGAS and STAT3 modulation compared to mono-functional dsISD⁴⁵-SNAs. (B) STAT3 reporter assay to monitor STAT3 transactivational activity. (C) IRF reporter assay in RAW-Lucia Macrophages. (D) Immunofluorescence microscopy showing subcellular distribution of phosphorylated STAT3. DAPI, nuclear counterstain. (E) Assessment of glioma stem cell sphere size upon treatment with different SNA architectures. (F) Kaplan Meyer survival curves of CT2A-bearing BL/6 mice treated via i.e. administration of indicated SNAs. Mean+/−standard deviations is shown. * p<0.05.

FIG. 10 shows the development of bimodal SNAs for concomitant cGAS activation and STAT3 inhibition. (A) Sequences of oligonucleotides conjugated to gold nanoparticle cores for cGAS activation and STAT3 inhibition. ssDNA45 oligonucleotides were used a negative control. Sequences: ssDNA^{T45}=5′-TACAGATCTACTAGTGATCTATGACTGATCTGTACATGATCTACA-3′ (SEQ ID NO: 5); ISD⁴⁵ Sense=5′-TACAGATCTACTAGTGATCTATGACTGATCTGTACATGATCTACA-3′ (SEQ ID NO: 10); ISD⁴⁵ Antisense=5′-ACATCTAGTACATGTCTAGTCAGTATCTAGTGATCATCAGACA-3′ (SEQ ID NO: 6); STAT3i Sense=5′-CTAAATGCCCTTTAC-3′ (SEQ ID NO: 7); STAT3i Antisense=5′-GTAAAGGGCATTTAG-3′ (SEQ ID NO: 8); ISD^{G5} STAT3i=5′-TACAACATTTCCCGTAAATCGGGGGGATTTA CGGGAAATGTTGTA-3′ (SEQ ID NO: 9) (B-E) Modified gel shift assays and Hill-Langmuir computing to assess SNA binding to human recombinant cGAS (B—C) and STAT3 (D-E). (F-G) Cell-free and cell-based IRF3 reporter assays demonstrating that all SNAs, i.e., ISD45-SNA, ISD^{G5}STAT3i, and STAT3i¹⁵ SNA architectures induced IRF3 activation. (H) IL6 reporter assay in HEK-Blue IL6 reporter cells, demonstrating that ISD¹⁵STAT3i and ISD^{G5}STAT3i-SNAs, but not ISD⁴⁵-SNAs, repressed STAT3-driven IL-6 transactivation.

FIG. 11 shows sexual dimorphism underlying anti-tumor effect of mono- or bimodal SNAs. (A) CT2A-bearing, (B) QPP7-bearing or (C) QPP4-bearing C57BL/6 male and female mice were treated with PBS, ssDNA45-SNA, ISD⁴⁵-SNAs, ISD¹⁵STAT3i or ISD^{G5}STAT3i-SNAs via intracranial administration (15 μmoles of Au), dosed on day 8 post-tumor cell implantation. Shown are Kaplan-Meier survival curves. P values were calculated using the log-rank (Mantel-Cox) test. *p<0.05.

FIG. 12 shows nanonstring expression profiling revealing immune cell activation in female but not in male QPP4 tumor-bearing animal subjects following SNA treatment. Expression profiling was performed using Nanostring nCounter PanCancer Immune Profiling Panel. Total RNA was isolated from tumor-infiltrating immune cells 30 days post tumor cell implantation. (A, B). Heatmap showing unsupervised clustering of z-score data for all the 730 genes for the treatment groups PBS, ssDNA45-SNA, ISD⁴⁵-SNAs, STAT3i¹⁵-SNAs, ISD^{G5}STAT3i-SNAs in female and male hosts. Selected genes listed in heatmaps represent distinct clusters that are responsible for immune activation or immune suppression. (C, D) Nanostring gene set pathway analysis for male and female immune cell populations. (E) Heatmap of z-score-ranked gene expression for ISD^{G5}STAT3i-SNAs treated male versus female mice (n=3 biological replicates). (F) Volcano plot of differentially expressed genes (Log2 FC>1.5 and p value <0.05) in females vs males for ISD^{G}S STAT3i-SNAs treatment groups. Key genes upregulated in females compared to males for the ISD^{G5}STAT3i-SNAs treatment are indicated. (G) Gene ontology analysis revealed enrichment of biological pathways in females compared to males for the ISD^{G5}STAT3i-SNAs treatment group. Data were analyzed using the ShinyGO tool (Ge S X, Jung D & Yao R, Bioinformatics 2020).

FIG. 13 shows that cGAS protein is expressed at higher levels in female versus male mice. (A) Western blot analysis of spleen and (B) BMDM lysates derived from male or female mice. β-actin is shown as a load control. (C) Flow cytometry-based quantification of cGAS protein level in tumor-associated myeloid cells (TAMCs) isolated from male or female PBS—, ssDNA45-SNA- or ISD⁴⁵-SNA-treated CT2A bearing mice.

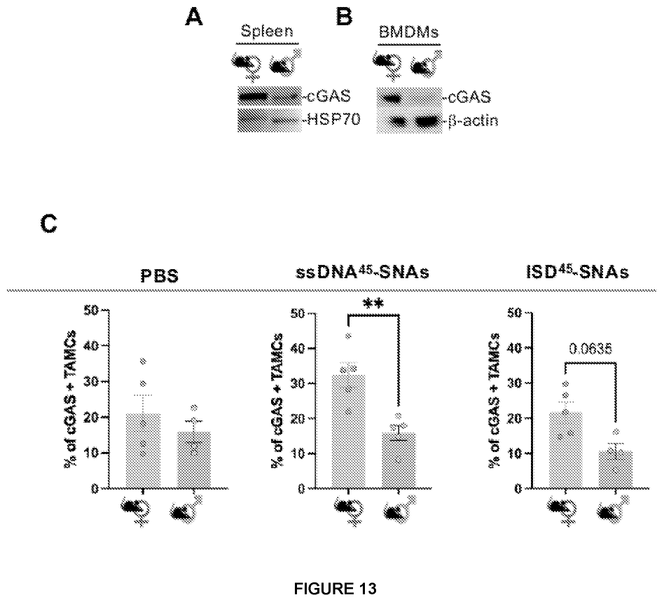

### DETAILED DESCRIPTION

Vaccines, drugs, and modified human cells that activate the immune system against tumor can improve the outcomes and prolong the lives of patients diagnosed with some type of cancers but have failed to provide survival benefits for patients with glioblastoma (GBM). Activation of the Stimulator of Interferon Genes (STING) pathway represents one of the main innate immune sensing pathway to enable natural killer (NK) and T cell priming against tumor.

The present disclosure demonstrates that the formulation of oligonucleotides into SNA structures (e.g., the presentation of oligonucleotides at high density on the surface of nanoparticles) leads to biochemical and biological properties that are radically different from those of linear (“free”) oligonucleotides. These properties include the cellular uptake of SNAs by a wide variety of cells, the gene regulatory activity of SNAs functionalized with siRNA or antisense DNA oligonucleotides, and the TLR-agonistic activity of SNAs conjugated with immunostimulatory oligonucleotides. Clinical trials with first generation siRNA-based SNAs (NCT03020017; GBM), and toll-like receptor 9 (TLR9)-agonistic SNAs (NCT03086278; solid cancers) have recently been completed.

Thus, in various aspects the present disclosure provides a cGAS agonistic immunotherapy by targeting cGAS—the sensor of cytosolic dsDNA upstream of STING—with SNAs presenting interferon-stimulating DNA (ISD) oligonucleotides at high surface density, and to evaluate the potential of SNAs^{D}, as a novel class of immunostimulatory therapy, for use in clinical neuro-oncology. This approach is distinct from other current approaches that target the STING pathway with small molecules (including CDNs). Without wishing to be bound by theory, by targeting cGAS, the strategy of using SNAs^{ISD} exploits the ability of cGAS to raise STING responses by delivering dsDNA and inducing the catalytic production of endogenous CDNs. The use of SNAs as provided herein addresses the challenges of delivery of therapeutic nucleic acids through the enhanced uptake of nucleic acids formulated as SNAs, and furthermore, exploits the polyvalent presentation of oligonucleotides at high density on a nanoparticle template. As shown herein, the binding of closely-spaced, neighboring dsDNA molecules on the surfaces of SNAs thus leads to potent cGAS activation. In some aspects, the disclosure provides bimodal cGAS-activating and STAT3 inhibitory SNAs capable of both eliciting potent type IFN responses and sequestering/inactivating STAT3 in the cytosol of cells to achieve effective anti-tumor immunity. In various embodiments, such bimodal SNAs comprise one or more cGAS-activating oligonucleotides, one or more oligonucleotides that inactivate STAT3, one or more oligonucleotides that activate cGAS and inactivate STAT3, or a combination thereof. In further aspects, the disclosure provides bimodal cytoplasmic DNA sensor-activating and transcription factor inhibitory SNAs capable of both eliciting potent type IFN responses and sequestering/inactivating transcription factor(s) (e.g., STAT3) in the cytosol of cells to achieve effective anti-tumor immunity. In various embodiments, such bimodal SNAs comprise one or more cytoplasmic DNA sensor-activating oligonucleotides, one or more oligonucleotides that inactivate a transcription factor (e.g., STAT3), one or more oligonucleotides that activate a cytoplasmic DNA sensor and inactivate a transcription factor, or a combination thereof.

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

The terms “polynucleotide” and “oligonucleotide” are interchangeable as used herein.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a SNA to a subject in need of treatment with such an agent. Such modes include, but are not limited to, oral, topical, intravenous, intraarterial, mucosal, intraperitoneal, intramuscular, intratumoral, parenteral, intradermal, intranasal, and subcutaneous administration. A combination of different routes of administration, separately or at the same time, is also contemplated by the disclosure, as is the combination of different routes of administration combined with focused ultrasound (FUS).

As used herein, “treating” and “treatment” refers to any reduction in the severity and/or onset of symptoms associated with a disorder. Accordingly, “treating” and “treatment” includes therapeutic and prophylactic measures. One of ordinary skill in the art will appreciate that any degree of protection from, or amelioration of, a disorder is beneficial to a subject, such as a human patient. The quality of life of a patient is improved by reducing to any degree the severity of symptoms in a subject and/or delaying or preventing the appearance of symptoms.

An “effective amount” or a “sufficient amount” of a substance is that amount necessary to effect beneficial or desired results, including clinical results, and, as such, an “effective amount” depends upon the context in which it is being applied. For example and without limitation, an effective amount of a SNA of the disclosure is the amount that is sufficient to elicit an immune response, inhibit gene expression, and/or treat a cancer. An effective amount can be administered in one or more doses. Efficacy can be shown in an experimental or clinical trial, for example, by comparing results achieved with a substance of interest compared to an experimental control.

The term “dose” as used herein in reference to a SNA of the disclosure refers to a measured portion of the SNA (e.g., as a pharmaceutical formulation) taken by (administered to or received by) a subject at any one time.

An “antigenic composition” is a composition of matter suitable for administration to a human or animal subject (e.g., in an experimental or clinical setting) that is capable of eliciting a specific immune response. In some embodiments, the immune response is elicited against an antigen, such as a cancer-related antigen. As such, in some embodiments an antigenic composition includes one or more antigens or antigenic epitopes.

An antigenic composition can, in some embodiments, also include one or more additional components capable of eliciting or enhancing an immune response, such as an excipient, carrier, and/or adjuvant.

An “immune response” is a response of the immune system (e.g., an interferon response) or of a cell of the immune system, such as a B cell, T cell, or monocyte, to a stimulus, such as an antigen (e.g., formulated as an antigenic composition). An immune response resulting from treatment with a SNA of the disclosure can trigger one or more of (1) CD8+ T cell infiltration and activation within the glioma tumor microenvironment; (2) M2/M0 to M1 re-education of macrophages; and (3) NK and NKT cells activation. An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”).

As used herein, an “immunostimulatory oligonucleotide” is an oligonucleotide that can stimulate (e.g., induce or enhance) an immune response (e.g., an interferon response). As described herein, the disclosure provides SNAs that comprise an immunostimulatory oligonucleotide that activates cGAS. In some embodiments, the disclosure provides SNAs that comprise an immunostimulatory oligonucleotide that activates a cytoplasmic DNA sensor. In some embodiments, a SNA of the disclosure comprises an oligonucleotide that activates cGAS and one or more additional immunostimulatory oligonucleotide(s) targeting additional DNA sensors or pattern recognition receptors. Typical examples of immunostimulatory oligonucleotides are CpG-motif containing oligonucleotides, single-stranded RNA oligonucleotides, double-stranded RNA oligonucleotides, a toll-like receptor (TLR) agonist, and double-stranded or single-stranded DNA or RNA oligonucleotides targeting additional DNA sensors, including but not limited to AIM2 (Absent in Melanoma-2), RNA polymerase Ill, DAI (DNA-dependent activator of IFN-regulatory factors), IFI16 (Interferon-γ-inducible protein 16), or a combination thereof. In various embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). A “CpG-motif” is a cytosine-guanine dinucleotide sequence. In various embodiments, one or more immunostimulatory oligonucleotides are encapsulated in the nanoparticle core of the SNA, attached to the external surface of the nanoparticle core, or a combination thereof. In some embodiments, one or more immunostimulatory oligonucleotides are encapsulated in the nanoparticle core and/or attached to the external surface of the nanoparticle core. In some embodiments, one or more immunostimulatory oligonucleotides are not associated with the SNA and are administered separately, either in the same composition as the SNA or in a separate composition.

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), single-guide RNA (sgRNA, in combination with Cas9 delivery), DNA or RNA inhibitors of innate immune receptors, an aptamer, a DNAzyme, or an aptazyme. In some embodiments, an inhibitory oligonucleotide is an oligonucleotide that binds to a receptor but does not activate the receptor, thereby inhibiting the receptor from further binding to a ligand and becoming activated.

All references, patents, and patent applications disclosed herein are incorporated by reference with respect to the subject matter for which each is cited, which in some cases may encompass the entirety of the document.

### Spherical Nucleic Acids (SNAs)

A “spherical nucleic acid” (SNA) as used herein comprises a spherical or substantially spherical nanoparticle core functionalized with a highly oriented oligonucleotide shell. In any of the aspects or embodiments of the disclosure, a SNA comprises (a) a nanoparticle core; and (b) a shell of oligonucleotides attached to the external surface of the nanoparticle core, the shell of oligonucleotides comprising one or more double-stranded and/or single-stranded stem loop DNA oligonucleotides that activates cyclic GMP-AMP synthase (cGAS) and are at least 15 base pairs in length. In some aspects, a SNA of the disclosure comprises (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that activates a cytoplasmic DNA sensor and is at least 15 base pairs in length. In any of the aspects or embodiments of the disclosure, a SNA comprises (a) a nanoparticle core; and (b) a shell of oligonucleotides attached to the external surface of the nanoparticle core, the shell of oligonucleotides comprising one or more double-stranded and/or single-stranded stem loop DNA oligonucleotides that (i) activates cyclic GMP-AMP synthase (cGAS); (ii) inactivates signal transducer and activator of transcription 3 (STAT3); and (iii) is at least 15 base pairs in length. In some aspects, a SNA of the disclosure comprises (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that (i) activates a cytoplasmic DNA sensor; (ii) inactivates a transcription factor; and (iii) is at least 15 base pairs in length. In some aspects, a SNA comprises (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which activates cyclic GMP-AMP synthase (cGAS) and is at least 15 base pairs in length. In some aspects, a SNA comprises (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which activates a cytoplasmic DNA sensor and is at least 15 base pairs in length. In some aspects, a SNA comprises (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which (i) activates cyclic GMP-AMP synthase (cGAS); (ii) inactivates signal transducer and activator of transcription 3 (STAT3); and (iii) is at least 15 base pairs in length. In some aspects, a SNA comprises (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which (i) activates a cytoplasmic DNA sensor; (ii) inactivates a transcription factor; and (iii) is at least 15 base pairs in length. In some aspects, a SNA comprises (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which (i) activates cyclic GMP-AMP synthase (cGAS) or other DNA sensors; (ii) inactivates signal transducer and activator of transcription 3 (STAT3) or other transcription factors; and (iii) is at least 15 base pairs in length.

In some aspects, the disclosure provides bimodal cGAS-activating and STAT3 inhibitory SNAs capable of both activating cGAS and inactivating STAT3. In various embodiments, such bimodal SNAs comprise one or more cGAS-activating oligonucleotides, one or more oligonucleotides that inactivate STAT3, one or more oligonucleotides that activate cGAS and inactivate STAT3, or a combination thereof. In some aspects, the disclosure provides bimodal cytoplasmic DNA sensor-activating and transcription factor inhibitory SNAs capable of both activating a cytoplasmic DNA sensor and inactivating the transcription factor. In various embodiments, such bimodal SNAs comprise one or more cytoplasmic DNA sensor-activating oligonucleotides, one or more oligonucleotides that inactivate a transcription actor, one or more oligonucleotides that activate a cytoplasmic DNA sensor and inactivate a transcription factor, or a combination thereof.

In some embodiments, the nanoparticle core is a protein core. In various embodiments, the protein core comprises or consists of a CRISPR-associated protein (e.g., Cas9). In some embodiments, a protein core comprising or consisting of a CRISPR-associated protein (e.g., Cas9) comprises one or more single guide RNAs (sgRNA) attached thereto.

In some embodiments, the nanoparticle core is a liposomal core. Liposomal cores of the disclosure have at least a substantially spherical geometry, an internal side and an external side, and comprise a lipid bilayer. The lipid bilayer comprises, in various embodiments, a plurality of lipid groups. The plurality of lipid groups comprises, in various embodiments, a lipid from the phosphatidylcholine, phosphatidylglycerol, and/or phosphatidylethanolamine families of lipids. While not meant to be limiting, in various embodiments the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), monophosphoryl Lipid A (MPLA), or a combination thereof.

The nanoparticle core of a SNA can range in size from about 1 nanometer (nm) to about 500 nm, about 1 nm to about 400 nm, about 1 nm to about 300 nm, about 1 nm to about 200 nm, about 1 nm to about 100 nm, about 1 nm to about 50 nm, about 10 nm to about 500 nm, about 10 nm to about 400 nm, about 10 nm to about 300 nm, about 10 nm to about 200 nm, about 10 nm to about 100 nm, about 10 nm to about 50 nm, about 10 nm to about 40 nm, about 20 nm to about 50 nm, about 20 nm to about 40 nm, about 20 nm to about 30 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In some embodiments, the nanoparticle core is a metallic core (e.g., gold) and is about 14 nm in diameter. In some embodiments, the nanoparticle core is a polymer core (e.g., PLGA) and is about 70 nm in diameter. In other aspects, the disclosure provides a plurality of SNAs, each comprising a nanoparticle core. In these aspects, the size of the plurality of nanoparticle cores is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, about 10 nm to about 20 nm in mean diameter, or about 20 nm to about 100 nm in mean diameter, about 20 nm to about 90 nm in mean diameter, about 20 nm to about 80 nm in mean diameter, about 20 nm to about 70 nm in mean diameter, about 20 nm to about 60 nm in mean diameter, about 20 nm to about 50 nm in mean diameter, about 20 nm to about 40 nm in mean diameter, or about 20 nm to about 30 nm in mean diameter. In some embodiments, the diameter (or mean diameter for a plurality of nanoparticle cores) of the nanoparticle cores is from about 10 nm to about 150 nm, from about 30 to about 100 nm, or from about 40 to about 80 nm. In some embodiments, the size of the nanoparticle cores used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs is produced and the SNAs in the plurality have a mean diameter of less than or equal to about 150 nanometers (e.g., about 10 nanometers to about 150 nanometers), or less than or equal to about 100 nanometers (e.g., about 10 nanometers to about 100 nanometers, or less than or equal to about 80 nanometers (e.g., about 10 nanometers to about 80 nanometers). In further embodiments, the nanoparticle cores in the plurality created by a method of the disclosure have a diameter or mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers, or less than or equal to about 50 nanometers, or less than or equal to about 55 nanometers, or less than or equal to about 60 nanometers, or less than or equal to about 65 nanometers, or less than or equal to about 70 nanometers, or less than or equal to about 75 nanometers, or less than or equal to about 80 nanometers, or less than or equal to about 85 nanometers, or less than or equal to about 90 nanometers, or less than or equal to about 95 nanometers, or less than or equal to about 100 nanometers, or less than or equal to about 100 nanometers, or less than or equal to about 120 nanometers, or less than or equal to about 130 nanometers, or less than or equal to about 140 nanometers, or less than or equal to about 150 nanometers. It will be understood that any of the foregoing diameters of nanoparticle cores can apply to the diameter of the nanoparticle core itself or to the diameter of the SNA (i.e., nanoparticle core and the shell of oligonucleotides attached to the external surface of the nanoparticle core).

As described herein, in some embodiments the nanoparticle core of the SNA is a liposomal core. Liposomal particles, for example as disclosed in International Patent Application No. PCT/US2014/068429 (incorporated by reference herein in its entirety) are therefore provided by the disclosure. Liposomal particles of the disclosure have at least a substantially spherical geometry, an internal side and an external side, and comprise a plurality of lipid groups. In various embodiments, the plurality of lipid groups comprises a lipid selected from the group consisting of the phosphatidylcholine, phosphatidylglycerol, and phosphatidylethanolamine families of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phospho-(1′-rac-glycerol) (DOPG), 1,2-distearoyl-sn-glycero-3-phosphoethanolamine (DSPE), cardiolipin, lipid A, monophosphoryl Lipid A (MPLA), or a combination thereof. In various embodiments, at least one oligonucleotide in the shell of oligonucleotides is attached to the external surface of the liposomal core through a lipid anchor group. In further embodiments, the lipid anchor group is attached to the 5′ end or the 3′ end of the at least one oligonucleotide. In still further embodiments, the lipid anchor group is tocopherol or cholesterol. Thus, in various embodiments, at least one of the oligonucleotides in the shell of oligonucleotides is an oligonucleotide-lipid conjugate containing a lipid anchor group, wherein said lipid anchor group is adsorbed into the lipid bilayer. In some embodiments, all of the oligonucleotides in the shell of oligonucleotides is an oligonucleotide-lipid conjugate containing a lipid anchor group, wherein said lipid anchor group is adsorbed into the lipid bilayer. In various embodiments, 10%, 20%, 30%, 40%, 50%, 60%, 70%, 80%, 90% or 100% of the oligonucleotides in the shell of oligonucleotides is attached (e.g., adsorbed) to the external surface of the liposomal core through a lipid anchor group. The lipid anchor group comprises, in various embodiments, tocopherol, palmitoyl, dipalmitoyl, stearyl, distearyl, or cholesterol. While not meant to be limiting, any chemistry known to one of skill in the art can be used to attach the lipid anchor to the oligonucleotide, including amide linking or click chemistry. Methods of making a liposomal SNA are generally known (see, e.g., Wang, S.; Qin, L.; Yamankurt, G.; Skakuj, K.; Huang, Z.; Chen, P.-C.; Dominguez, D.; Lee, A.; Zhang, B.; Mirkin, C. A. Rational Vaccinology with Spherical Nucleic Acids. Proc. Natl. Acad. Sci. 2019, 116 (21), 10473-10481, incorporated by reference herein in its entirety).

In some embodiments, the nanoparticle core is a lipid nanoparticle core. Lipid nanoparticle (LNP) spherical nucleic acids comprise a lipid nanoparticle core (e.g., a solid lipid nanoparticle core) decorated with a shell of oligonucleotides. The lipid nanoparticle core comprises an ionizable lipid, a phospholipid, a sterol, and a lipid-polyethylene glycol (lipid-PEG) conjugate. The shell of oligonucleotides is attached to the external surface of the lipid nanoparticle core, and in any of the aspects or embodiments of the disclosure comprises one or more cGAS-activating oligonucleotides. In some embodiments, the shell of oligonucleotides attached to the external surface of the lipid nanoparticle core comprises one or more oligonucleotides that activate cGAS and inactivate STAT3. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

In various embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), monophosphoryl Lipid A (MPLA), or a combination thereof. In further embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (R-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

In any of the aspects or embodiments of the disclosure an oligonucleotide is attached to the exterior of a lipid nanoparticle core via a covalent attachment of the oligonucleotide to a lipid-polyethylene glycol (lipid-PEG) conjugate. In various embodiments, 10%, 20%, 30%, 40%, 50%, 60%, 70%, 80%, 90% or 100% of the oligonucleotides in the shell of oligonucleotides are covalently attached to the exterior of the lipid nanoparticle core through the lipid-PEG conjugate. In various embodiments, one or more oligonucleotides in the oligonucleotide shell is attached (e.g., adsorbed) to the exterior of the lipid nanoparticle core through a lipid anchor group as described herein. In various embodiments, 10%, 20%, 30%, 40%, 50%, 60%, 70%, 80%, 90% or 100% of the oligonucleotides in the shell of oligonucleotides is attached (e.g., adsorbed) to the exterior of the lipid nanoparticle core through a lipid anchor group as described herein. The lipid anchor group is, in various embodiments, attached to the 5′- or 3′-end of the oligonucleotide. In various embodiments, the lipid anchor group is tocopherol, palmitoyl, dipalmitoyl, stearyl, distearyl, or cholesterol.

### Oligonucleotides

In some aspects, the disclosure provides spherical nucleic acids (SNAs) comprising (a) a nanoparticle core; and (b) a shell of oligonucleotides attached to the external surface of the nanoparticle core, the shell of oligonucleotides comprising a double-stranded or single-stranded stem loop DNA oligonucleotide that activates cyclic GMP-AMP synthase (cGAS) and is at least 15 base pairs in length. In some aspects, the disclosure provides spherical nucleic acids (SNAs) comprising (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that activates a cytoplasmic DNA sensor and is at least 15 base pairs in length. In some aspects, the disclosure provides spherical nucleic acids (SNAs) comprising (a) a nanoparticle core; and (b) a shell of oligonucleotides attached to the external surface of the nanoparticle core, the shell of oligonucleotides comprising one or more double-stranded and/or single-stranded stem loop DNA oligonucleotides that activates a cytoplasmic DNA sensor and is at least 15 base pairs in length. In various embodiments, the cytoplasmic DNA sensor is cyclic GMP-AMP synthase (cGAS), AIM2 (Absent in Melanoma-2), RNA polymerase Ill, DAI (DNA-dependent activator of IFN-regulatory factors), IFI16 (Interferon-γ-inducible protein 16), or a combination thereof. In some aspects, the disclosure provides spherical nucleic acids (SNAs) comprising (a) a nanoparticle core; and (b) a shell of oligonucleotides attached to the external surface of the nanoparticle core, the shell of oligonucleotides comprising one or more double-stranded and/or single-stranded stem loop DNA oligonucleotides that activates cyclic GMP-AMP synthase (cGAS) and is at least 15 base pairs in length. In some aspects, the disclosure provides spherical nucleic acids (SNAs) comprising (a) a nanoparticle core; and (b) a shell of oligonucleotides attached to the external surface of the nanoparticle core, the shell of oligonucleotides comprising one or more double-stranded and/or single-stranded stem loop DNA oligonucleotides that (i) activates cyclic GMP-AMP synthase (cGAS); (ii) inactivates signal transducer and activator of transcription 3 (STAT3); and (iii) is at least 15 base pairs in length. In some aspects, the disclosure provides spherical nucleic acids (SNAs) comprising (a) a nanoparticle core; and (b) a shell of oligonucleotides attached to the external surface of the nanoparticle core, the shell of oligonucleotides comprising one or more double-stranded and/or single-stranded stem loop DNA oligonucleotides that (i) activates a cytoplasmic DNA sensor; (ii) inactivates a transcription factor; and (iii) is at least 15 base pairs in length. In any of the aspects or embodiments of the disclosure, the transcription factor promotes cancer progression. In various embodiments, the transcription factor is signal transducer and activator of transcription 3 (STAT3), cMyc, NANOG, SOX2, OCT4, or a combination thereof. In some embodiments, the shell of oligonucleotides comprises a plurality of double-stranded and/or single-stranded stem loop DNA oligonucleotides, each of which activates cGAS and is at least 15 base pairs in length. In some embodiments, the shell of oligonucleotides comprises a plurality of double-stranded and/or single-stranded stem loop DNA oligonucleotides, each of which activates cGAS, inactivates STAT3, and is at least 15 base pairs in length. In further embodiments, the shell of oligonucleotides consists of a plurality of double-stranded and/or single-stranded stem loop DNA oligonucleotides, each of which activates cGAS and is at least 15 base pairs in length. In still further embodiments, the shell of oligonucleotides consists of a plurality of double-stranded and/or single-stranded stem loop DNA oligonucleotides, each of which activates cGAS, inactivates STAT3, and is at least 15 base pairs in length. In some embodiments, the shell of oligonucleotides comprises a plurality of double-stranded and/or single-stranded stem loop DNA oligonucleotides, each of which activates a cytoplasmic DNA sensor and is at least 15 base pairs in length. In some embodiments, the shell of oligonucleotides comprises a plurality of double-stranded and/or single-stranded stem loop DNA oligonucleotides, each of which activates a cytoplasmic DNA sensor, inactivates a transcription factor, and is at least 15 base pairs in length. In further embodiments, the shell of oligonucleotides consists of a plurality of double-stranded and/or single-stranded stem loop DNA oligonucleotides, each of which activates a cytoplasmic DNA sensor and is at least 15 base pairs in length. In still further embodiments, the shell of oligonucleotides consists of a plurality of double-stranded and/or single-stranded stem loop DNA oligonucleotides, each of which activates a cytoplasmic DNA sensor, inactivates a transcription factor, and is at least 15 base pairs in length. In various embodiments, the shell of oligonucleotides comprises one or more additional oligonucleotides. In some embodiments, a SNA of the disclosure comprises one or more additional oligonucleotides encapsulated in the nanoparticle core. In some embodiments, the shell of oligonucleotides comprises one or more additional oligonucleotides and one or more additional oligonucleotides is encapsulated in the nanoparticle core. In some embodiments, the one or more additional oligonucleotides each have the same nucleotide sequence. In some embodiments, the one or more additional oligonucleotides comprises at least two oligonucleotides that have a different nucleotide sequence. In various embodiments, the additional oligonucleotide is an immunostimulatory oligonucleotide, an inhibitory oligonucleotide, an oligonucleotide that inactivates signal transducer and activator of transcription 3 (STAT3), or a combination thereof. Oligonucleotides in the shell of oligonucleotides may be attached to a nanoparticle core through any means (e.g., covalent or non-covalent attachment). An oligonucleotide in the shell of oligonucleotides may be attached to a nanoparticle core via its 5′ end or 3′ end.

An oligonucleotide that “activates cyclic GMP-AMP synthase (cGAS)” or a “cGAS-activating oligonucleotide” or the like refers to an oligonucleotide that can activate cGAS and lead to activation of Stimulator of Interferon Genes (STING), downstream IRF3 and NF-κB signaling, and the release of proinflammatory mediators and type-I interferon (IFN). Such oligonucleotides generally comprise double-stranded DNA oligonucleotides or single-stranded stem loop DNA oligonucleotides that are at least about 15 base pairs (bp) in length, and optionally do not contain CpG-rich motifs. Thus, in any of the aspects or embodiments of the disclosure, the shell of oligonucleotides on the surface of a SNA comprises one or more oligonucleotides that activate cGAS, wherein the one or more oligonucleotides comprise double-stranded DNA oligonucleotides, including G-quartet oligonucleotides, single-stranded stem loop DNA oligonucleotides, or a combination thereof. In some embodiments, the shell of oligonucleotides on the surface of a SNA comprises a plurality of oligonucleotides that activate cGAS, wherein the plurality of oligonucleotides comprises double-stranded DNA oligonucleotides, single-stranded stem loop DNA oligonucleotides, or a combination thereof. In some embodiments, one or more or all oligonucleotides on the external surface of a SNA activates cGAS and inactivates STAT3. Assays for identifying cGAS-activating oligonucleotides are known in the art (e.g., Transcreener® assay (BellBrook Labs, Madison, WI; see also Herzner A M. et al., 2015. Sequence-specific activation of the DNA sensor cGAS by Y-form DNA structures as found in primary HIV-1 cDNA. Nat Immunol. 16(10):1025-33) and are described herein.

An oligonucleotide that “inactivates signal transducer and activator of transcription 3 (STAT3)” is one that can inactivate or inhibit the transcription factor STAT3. In various embodiments, an oligonucleotide that inactivates STAT3 is a transcription factor decoy, a G-quartet oligonucleotide, an aptamer, an inhibitory oligonucleotide, or a combination thereof. Transcription factor decoys include nucleotide sequences derived from conserved genomic regulatory elements that are recognized and bound by the transcription factor in question (i.e., STAT3). The decoys act by competitively inhibiting binding of the transcription factor to corresponding cis elements in genomic DNA, preventing expression of target genes. Assays for identifying an oligonucleotide that can inactivate STAT3 are generally known in the art (e.g., Leong P L, Andrews G A, Johnson D E, et al. Targeted inhibition of Stat3 with a decoy oligonucleotide abrogates head and neck cancer cell growth. Proc Natl Acad Sci USA. 2003; 100(7):4138-4143. doi:10.1073/pnas.0534764100; K. Lau Y-T, Ramaiyer M, E. Johnson D, R. Grandis J. Targeting STAT3 in Cancer with Nucleotide Therapeutics. Cancers. 2019; 11(11):1681). A non-limiting example of a STAT3 decoy that can be used according to the disclosure is as follows:

STAT3 Decoy

| | |
|---|---|
| Sense | 5′-CATTTCCCGTAAATC-3′ (SEQ ID NO: 1) |
| Antisense | 3′ GTAAAGGGCATTTAG 5′ (SEQ ID NO: 2) |

Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA).

In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C=NR^{H}, >C=S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}— CH₂—, —CH₂—NR^{H}—O—, —CH₂—NR^{H}—CO—, —O—NR^{H}— CH₂—, —O—NR^{H}, —O— CH₂—S—, —S— CH₂—O—, —CH₂— CH₂—S—, —O— CH₂— CH₂—S—, —S—CH₂—CH=(including R⁵ when used as a linkage to a succeeding monomer), —S—CH₂— CH₂—, —S— CH₂— CH₂—O—, —S— CH₂— CH₂—S—, —CH₂—S— CH₂—, —CH₂—SO— CH₂—, —CH₂—SO₂— CH₂—, —O—SO—O—, —O—S(O)₂-O—, —O—S(O)₂— CH₂—, —O—S(O)₂—NR^{H}—, —NR^{H}_S(O)₂— CH₂—; —O—S(O)₂— CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—, —O—P(O)₂—NR^{H}H—, —NR^{H}—P(O)₂—O—, —O—P(O,NR^{H})—CH₂—P(O)₂—O—, —O—P(O)₂— CH₂—, and —O—Si(R″)₂—O—; among which —CH₂—CO—NR^{H}—, —CH₂—NR^{H}—O—, —S— CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]2, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1 H-pyrimido[5,4-b][1,4]benzoxazin-2(3H)-one), phenothiazine cytidine (1 H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., a cGAS-activating oligonucleotide), or a modified form thereof, is generally about 5 nucleotides to about 5000 nucleotides in length. In various embodiments, an oligonucleotide of the disclosure is about 5 to about 4000 nucleotides in length, about 5 to about 3000 nucleotides in length, about 5 to about 2000 nucleotides in length, about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 4000 nucleotides in length, about 10 to about 3000 nucleotides in length, about 10 to about 2000 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, 2000, 3000, 4000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1500, 2000, 2500, 3000, 4000, or more nucleotides in length. In further embodiments, an oligonucleotide of the disclosure is about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 15 to about 250 nucleotides in length, about 15 to about 200 nucleotides in length, about 15 to about 150 nucleotides in length, about 15 to about 100 nucleotides in length, about 15 to about 90 nucleotides in length, about 15 to about 80 nucleotides in length, about 15 to about 70 nucleotides in length, about 15 to about 60 nucleotides in length, about 15 to about 50 nucleotides in length about 15 to about 45 nucleotides in length, about 15 to about 40 nucleotides in length, about 15 to about 35 nucleotides in length, about 15 to about 30 nucleotides in length, about 15 to about 25 nucleotides in length, about 15 to about 20 nucleotides in length, and all oligonucleotides intermediate in length of the sizes specifically disclosed to the extent that the oligonucleotide is able to achieve the desired result. In some embodiments, an oligonucleotide of the disclosure is or is at least 15 nucleotides in length. It will be understood that each of the foregoing lengths can refer to the length in nucleotides (for example, when referring to single-stranded nucleic acids) or the length in base pairs (for example, when referring to double-stranded nucleic acids such as cGAS-activating oligonucleotides). In various embodiments, the shell of oligonucleotides attached to the external surface of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. In some embodiments, the shell of oligonucleotides comprises a plurality of double-stranded and/or single-stranded stem loop DNA oligonucleotides, each of which activates cGAS, or other cytoplasmic DNA sensors, including but not limited to AIM2 (Absent in Melanoma-2), RNA polymerase Ill, DAI (DNA-dependent activator of IFN-regulatory factors), or IFI16 (Interferon-γ-inducible protein 16, and is at least 15 base pairs in length. In some embodiments, the shell of oligonucleotides comprises a plurality of double-stranded and/or single-stranded stem loop DNA oligonucleotides, each of which activates cGAS and is at least 15 base pairs in length and further comprises one or more additional oligonucleotides. In some embodiments, the shell of oligonucleotides consists of a plurality of double-stranded and/or single-stranded stem loop DNA oligonucleotides, each of which activates cGAS and is at least 15 base pairs in length. In some embodiments, one or more oligonucleotides in the shell of oligonucleotides is a dual-function oligonucleotide that activates cGAS and also inactivates signal transducer and activator of transcription 3 (STAT3). In some embodiments, one or more oligonucleotides in the shell of oligonucleotides is a dual-function oligonucleotide that activates a cytoplasmic DNA sensor (for example and without limitation cyclic GMP-AMP synthase (cGAS), AIM2 (Absent in Melanoma-2), RNA polymerase Ill, DAI (DNA-dependent activator of IFN-regulatory factors), IFI16 (Interferon-γ-inducible protein 16), or a combination thereof) and also inactivates a transcription factor (for example and without limitation signal transducer and activator of transcription 3 (STAT3), cMyc, NANOG, SOX2, OCT4, or a combination thereof). In some embodiments, the shell of oligonucleotides comprises (i) one or more double-stranded and/or single-stranded stem loop DNA oligonucleotides, each of which activates cyclic GMP-AMP synthase (cGAS) and is at least 15 base pairs in length and (ii) one or more oligonucleotides that inactivate signal transducer and activator of transcription 3 (STAT3). In various embodiments, a SNA of the disclosure comprises a shell of oligonucleotides comprising (i) one or more double-stranded and/or single-stranded stem loop DNA oligonucleotides that activate cyclic GMP-AMP synthase (cGAS) and is at least 15 base pairs in length; (ii) one or more oligonucleotides that inactivates signal transducer and activator of transcription 3 (STAT3); (iii) one or more oligonucleotides, each of which activates cGAS and inactivates signal transducer and activator of transcription 3 (STAT3); or (iv) a combination thereof. In some embodiments, the shell of oligonucleotides comprises (i) one or more double-stranded and/or single-stranded stem loop DNA oligonucleotides, each of which activates a cytoplasmic DNA sensor and is at least 15 base pairs in length and (ii) one or more oligonucleotides that inactivate a transcription factor. In various embodiments, a SNA of the disclosure comprises a shell of oligonucleotides comprising (i) one or more double-stranded and/or single-stranded stem loop DNA oligonucleotides that activate a cytoplasmic DNA sensor and is at least 15 base pairs in length; (ii) one or more oligonucleotides that inactivates a transcription factor; (iii) one or more oligonucleotides, each of which activates a cytoplasmic DNA sensor and inactivates a transcription factor; or (iv) a combination thereof.

Spacers. In some aspects and embodiments, one or more oligonucleotides in the shell of oligonucleotides that is attached to the nanoparticle core of a SNA comprise a spacer. In some aspects and embodiments, each oligonucleotide in the shell of oligonucleotides that is attached to the nanoparticle core of a SNA comprises a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core. In some embodiments, an oligonucleotide encapsulated in the nanoparticle core of a SNA comprises a spacer.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

Density. The number of oligonucleotides in the shell of oligonucleotides on the external surface of a SNA can vary. In general, a surface density adequate to make the nanoparticles stable and the conditions necessary to obtain it for a desired combination of nanoparticles and oligonucleotides can be determined empirically. In some embodiments, one or more oligonucleotides is also encapsulated in the nanoparticle core. The number and length of oligonucleotides encapsulated in a nanoparticle core may be determined to an extent by the diameter of the nanoparticle core. Similarly, the number of oligonucleotides on the external surface of a SNA may be determined to an extent by the diameter of the nanoparticle core and/or the nucleotide sequence of the oligonucleotide. Generally, a surface density of at least about 2 pmoles/cm² will be adequate to provide stable nanoparticle-oligonucleotide compositions. In some aspects, the surface density is or is at least 15 pmoles/cm². Methods are also provided wherein the oligonucleotide is attached to the external surface of the nanoparticle core at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of oligonucleotide on the external surface of the SNA is measured by the number of oligonucleotides on the external surface of a SNA. With respect to the surface density of oligonucleotides on the external surface of a SNA of the disclosure, it is contemplated that a SNA as described herein comprises about 1 to about 250 oligonucleotides on its external surface. In various embodiments, a SNA comprises about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, about 10 to about 20, or about 50 to about 200, or about 50 to about 150, or about 50 to about 100, or about 50 to about 80 oligonucleotides on its surface. In further embodiments, a SNA comprises about 4 to about 250, or about 4 to about 200, or about 4 to about 190, or about 4 to about 180, or about 4 to about 170, or about 4 to about 160, or about 4 to about 150, or about 4 to about 140, or about 4 to about 130, or about 4 to about 120, or about 4 to about 110, or about 4 to about 100, or 4 to about 90, or about 4 to about 80, or about 4 to about 70, or about 4 to about 60, or about 4 to about 50, or about 4 to about 40, or about 4 to about 30, or about 4 to about 20, or about 4 to about 10, or about 50 to about 200, or about 50 to about 150, or about 50 to about 100, or about 50 to about 80 oligonucleotides on its surface. In some embodiments, a SNA comprises about 80 to about 140 oligonucleotides on its surface. In further embodiments, a SNA comprises at least about 5, 10, 20, 30, 40, 45, 50, 55, 60, 65, 70, 75, 80, 85, 90, 95, 100, 105, 110, 115, 120, 125,130, 135, 140, 145, 150, 155, 160, 165, 170,175, 180, 185, 190, 195, 200, 210, 220, 230, 240, or 250 polynucleotides on its surface. In further embodiments, a SNA consists of 5, 10, 20, 30, 40, 45, 50, 55, 60, 65, 70, 75, 80, 85, 90, 95, 100, 105, 110, 115, 120, 125,130, 135, 140, 145, 150, 155, 160, 165, 170,175, 180, 185, 190, 195, 200, 210, 220, 230, 240, or 250 polynucleotides on its surface. In some embodiments, a SNA comprising a liposomal or lipid nanoparticle core (which may, in various embodiments, be about or less than about 150 nanometers in diameter or about or less than about 100 nanometers in diameter or about or less than about 80 nanometers in diameter or about or less than about 70 nanometers in diameter) comprises about 10 to about 2,000 oligonucleotides, or about 10 to about 1,000 oligonucleotides, or about 10 to about 100 oligonucleotides, or about 10 to about 80 oligonucleotides, or about 10 to about 40 oligonucleotides on its surface. In some embodiments, a SNA comprising a metallic (e.g., gold) core comprises about 70 to about 120 oligonucleotides on its surface. In some embodiments, a SNA comprising a polymer (e.g., PLGA) core comprises about 70 oligonucleotides on its surface.

### Compositions

In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising one or more double-stranded and/or single-stranded stem loop DNA oligonucleotide that activates cyclic GMP-AMP synthase (cGAS) and is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising a double-stranded DNA oligonucleotide or single-stranded stem loop DNA oligonucleotide that activates a cytoplasmic DNA sensor and is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising one or more double-stranded and/or single-stranded stem loop DNA oligonucleotides that (i) activates cyclic GMP-AMP synthase (cGAS); (ii) inactivates signal transducer and activator of transcription 3 (STAT3); and (iii) is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising one or more double-stranded and/or single-stranded stem loop DNA oligonucleotides that (i) activates a cytoplasmic DNA sensor; (ii) inactivates a transcription factor; and (iii) is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which activates cyclic GMP-AMP synthase (cGAS) and is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which (i) activates cyclic GMP-AMP synthase (cGAS); (ii) inactivates signal transducer and activator of transcription 3 (STAT3); and (iii) is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which activates a cytoplasmic DNA sensor and is at least 15 base pairs in length. In some aspects, the disclosure provides compositions that comprise one or a plurality of spherical nucleic acid (SNA) comprising: (a) a nanoparticle core; and (b) a shell of oligonucleotides attached to the external surface of the nanoparticle core, the shell of oligonucleotides comprising a plurality of double-stranded and/or single-stranded stem loop DNA oligonucleotides, each of which (i) activates a cytoplasmic DNA sensor; (ii) inactivates a transcription factor; and (iii) is at least 15 base pairs in length. In various embodiments, the cytoplasmic DNA sensor is cyclic GMP-AMP synthase (cGAS), AIM2 (Absent in Melanoma-2), RNA polymerase Ill, DAI (DNA-dependent activator of IFN-regulatory factors), IFI16 (Interferon-γ-inducible protein 16), or a combination thereof. In any of the aspects or embodiments of the disclosure, the transcription factor promotes cancer progression. In various embodiments, the transcription factor is signal transducer and activator of transcription 3 (STAT3), cMyc, NANOG, SOX2, OCT4, or a combination thereof.

In some embodiments, the composition is an antigenic composition. In some embodiments, the composition further comprises a pharmaceutically acceptable carrier. The term “carrier” refers to a vehicle within which the SNA as described herein is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances that may enhance vaccine stability and include but are not limited to polymers (e.g., polyethylene glycol), carbohydrates (e.g., starch, glucose, lactose, sucrose, or cellulose), and alcohols (e.g., glycerol, sorbitol, or xylitol). Adjuvants include vaccine delivery systems (e.g., emulsions, microparticles, immune stimulating complexes (ISCOMS), or liposomes) that target associated antigens to antigen presenting cells (APC); and immunostimulatory adjuvants.

### Antigens

In various aspects, the disclosure provides spherical nucleic acids (SNAs) comprising (a) a nanoparticle core; and (b) a shell of oligonucleotides attached to the external surface of the nanoparticle core, the shell of oligonucleotides comprising a double-stranded or single-stranded stem loop DNA oligonucleotide that activates cyclic GMP-AMP synthase (cGAS) and is at least 15 base pairs in length. In various aspects, the disclosure provides spherical nucleic acids (SNAs) comprising (a) a nanoparticle core; and (b) a shell of oligonucleotides attached to the external surface of the nanoparticle core, the shell of oligonucleotides comprising a double-stranded or single-stranded stem loop DNA oligonucleotide that activates a cytoplasmic DNA sensor as described herein and is at least 15 base pairs in length. In some embodiments, a SNA of the disclosure comprises an antigen. In various embodiments, the antigen is a peptide, a protein, a DNA or RNA molecule encoding an antigen, or a combination thereof. In some embodiments, the antigen is a cancer-related antigen. In further embodiments, the antigen is a tumor associated antigen, a tumor specific antigen, a neo-antigen, or a combination thereof. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In various embodiments, the antigen is OVA1, MSLN, P53, Ras, mutant IDH1 (IDH1R132H), mutant telomerase reverse transcriptase (TERT; C228T or C250T), a melanoma related antigen, a HPV related antigen, a prostate cancer related antigen, a glioblastoma antigen, a grade IV astrocytoma antigen, an ovarian cancer related antigen, a breast cancer related antigen, a hepatocellular carcinoma related antigen, a bowel cancer related antigen, or human papillomavirus (HPV) E7 nuclear protein.

In some embodiments, the antigen is encapsulated in the nanoparticle core. In some embodiments, the antigen is a DNA or RNA molecule encoding the antigen that is encapsulated in the nanoparticle core. In some embodiments, the antigen is attached to the surface of the nanoparticle core, the antigen is attached to an oligonucleotide in the shell of oligonucleotides, or both (see, e.g., U.S. Patent Application Publication No. 2020/0384104, incorporated herein by reference in its entirety). In further embodiments, an antigen is both (i) encapsulated in the nanoparticle core and (ii) attached to the surface of the nanoparticle core and/or attached to an oligonucleotide in the shell of oligonucleotides. In some embodiments, the antigen is attached to the surface of the nanoparticle core via a linker.

### Uses of SNAs to Treat a Disorder

In some embodiments, a SNA of the disclosure is used to treat, attenuate, or ameliorate a disorder (e.g., cancer). Thus, in some aspects, the disclosure provides methods of treating or ameliorating a disorder (e.g., a cancer) comprising administering an effective amount of a SNA or composition of the disclosure to a subject (e.g., a human subject) in need thereof, wherein the administering treats or ameliorates the disorder. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

Administration of SNAs and compositions of the disclosure can involve a single dose or a multiple dose schedule. In a multiple dose schedule the various doses may be given by the same or different routes.

### Methods of Gene Regulation

In some aspects of the disclosure, an oligonucleotide associated with a SNA of the disclosure inhibits the expression of a gene. Thus, in some embodiments, a SNA of the disclosure activates cGAS, optionally inactivates STAT3, and also has a gene inhibitory function. Accordingly, in some embodiments the shell of oligonucleotides that is attached to the external surface of the nanoparticle core comprises one or more double-stranded and/or single-stranded stem loop DNA oligonucleotides that activate cyclic GMP-AMP synthase (cGAS) and are each at least 15 base pairs in length, and further comprises one or more inhibitory oligonucleotides designed to inhibit target gene expression. In some embodiments, a SNA of the disclosure activates a cytoplasmic DNA sensor, optionally inactivates a transcription factor, and also has a gene inhibitory function. In further embodiments, the shell of oligonucleotides that is attached to the external surface of the nanoparticle core comprises one or more double-stranded and/or single-stranded stem loop DNA oligonucleotides that activate a cytoplasmic DNA sensor as described herein and are each at least 15 base pairs in length, and further comprises one or more inhibitory oligonucleotides designed to inhibit target gene expression. In some embodiments, one or more inhibitory oligonucleotides designed to inhibit target gene expression is encapsulated in the nanoparticle core. In some embodiments, one or more inhibitory oligonucleotides designed to inhibit target gene expression is both attached to the external surface of the nanoparticle core and encapsulated in the nanoparticle core.

Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), single guide RNA (sgRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

### Therapeutic Agents

The technology provided herein has broad applicability. SNAs of the disclosure (e.g., SNAs^{ISD} and SNAs^{ISD/STAT3i}) are a class of oligonucleotide-based drugs that can be used as generalizable anti-neoplastic agents against a broad spectrum of tumor types.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

In some embodiments a SNA is used in combination with one or more standard-of-care therapeutic agents, such as radiation, and extant immunotherapies, including but not limited to checkpoint inhibitors, such as anti-PD1, and inhibitors of immunosuppressive adenosine signaling, such as anti-CD73 (Ty/23) and the A2aR inhibitor SCH58261.

Thus, in some embodiments, an SNA of the disclosure further comprises a therapeutic agent, or a plurality thereof. The therapeutic agent is, in various embodiments, simply associated with an oligonucleotide in the shell of oligonucleotides attached to the external surface of the nanoparticle core of the SNA, and/or the therapeutic agent is associated with the nanoparticle core of the SNA, and/or the therapeutic agent is encapsulated in the SNA. In some embodiments, the therapeutic agent is associated with the end of an oligonucleotide in the shell of oligonucleotides that is not attached to the nanoparticle core (e.g., if the oligonucleotide is attached to the nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the nanoparticle core (e.g., if the oligonucleotide is attached to the nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the external surface of the nanoparticle core of the SNA. In some embodiments, the therapeutic agent is covalently associated with a linker or spacer that is attached to the external surface of the nanoparticle core of the SNA. In some embodiments, the therapeutic agent is non-covalently associated with an oligonucleotide in the shell of oligonucleotides that is attached to the external surface of the nanoparticle core of the SNA. However, it is understood that the disclosure provides SNAs wherein one or more therapeutic agents are both covalently and non-covalently associated with oligonucleotides in the shell of oligonucleotides that is attached to the external surface of the nanoparticle core of the SNA. It will also be understood that non-covalent associations include hybridization, protein binding, and/or hydrophobic interactions. In some embodiments, a therapeutic agent is administered separately from a SNA of the disclosure. Thus, in some embodiments, a therapeutic agent is administered before, after, or concurrently with a SNA of the disclosure to treat a disorder.

The following examples illustrate various embodiments contemplated by the present disclosure. The examples are exemplary in nature and are in no way intended to be limiting.

### EXAMPLES

### Example 1

In this Example, cGAS-activating SNAs, which present interferon-stimulatory double stranded (ds)DNA (ISD) at high surface density on gold nanoparticle cores (SNAs^{ISD}) were synthesized and characterized. The data provided herein demonstrated that SNAs^{ISD} activated the cGAS-STING pathway more potently than CDNs, culminating in the downstream induction of IRF and NF-κB-dependent transcription. When intratumorally or intranasally administered to immunocompetent GBM-bearing mice, SNAs^{ISD} antagonized tumor progression more robustly than free ISD⁴⁵ oligonucleotides or CDNs and promoted long-term animal subject survival through specific activation of the cGAS-STING signaling pathway.

In addition to monofunctional cGAS-activating SNAs, bimodal SNAs capable of activating cGAS and concomitantly inhibiting the transactivational activity of the STAT3 master transcription factor were generated. As DNA recognition by cGAS occurs independent of oligonucleotide sequence, to this end, canonical ISD DNA oligonucleotides were replaced with oligonucleotides shown to bind to, sequester and inactivate the master transcription factor STAT3. It was demonstrated that bimodal SNAs^{ISD/STAT3i} SNAs activated cGAS and blocked nuclear translocation as well as transcriptional activity of STAT3 in vitro and antagonized glioma progression in vivo.

### Development and Characterization of ISD⁴⁵-SNAs

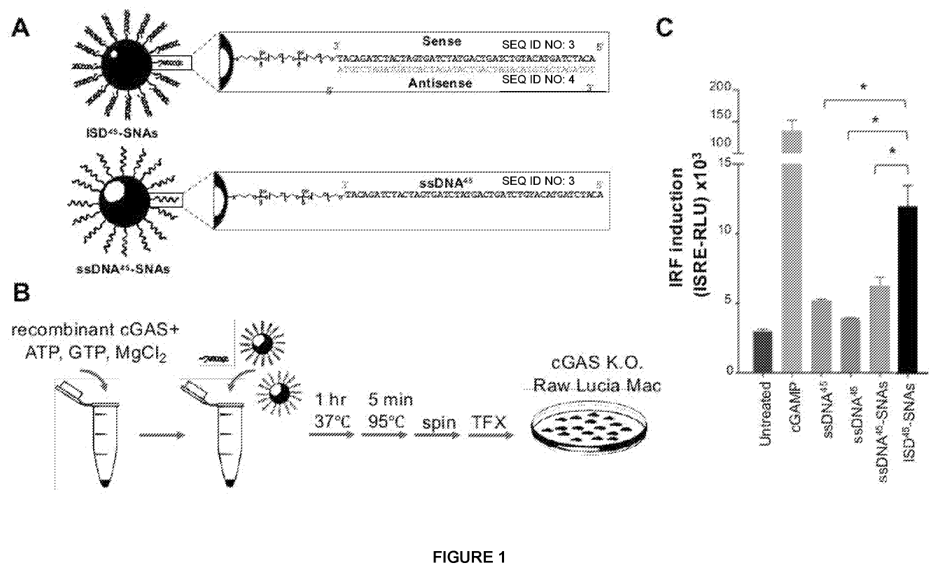

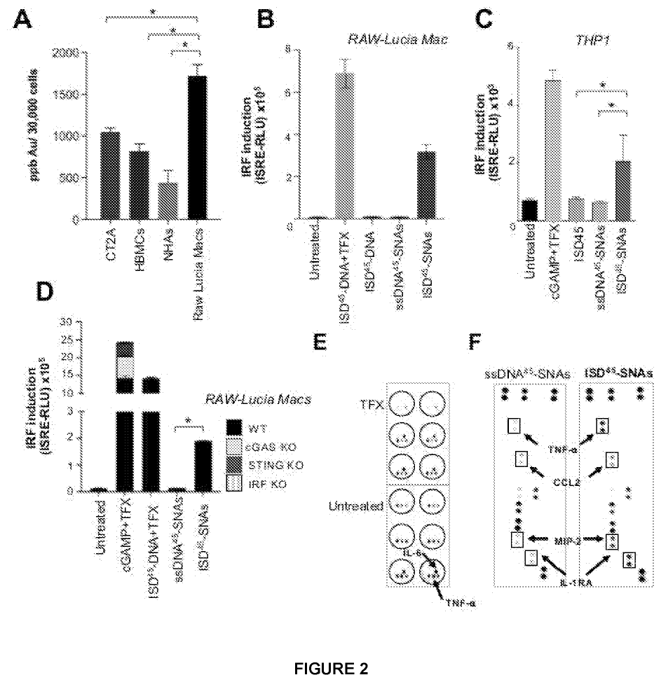

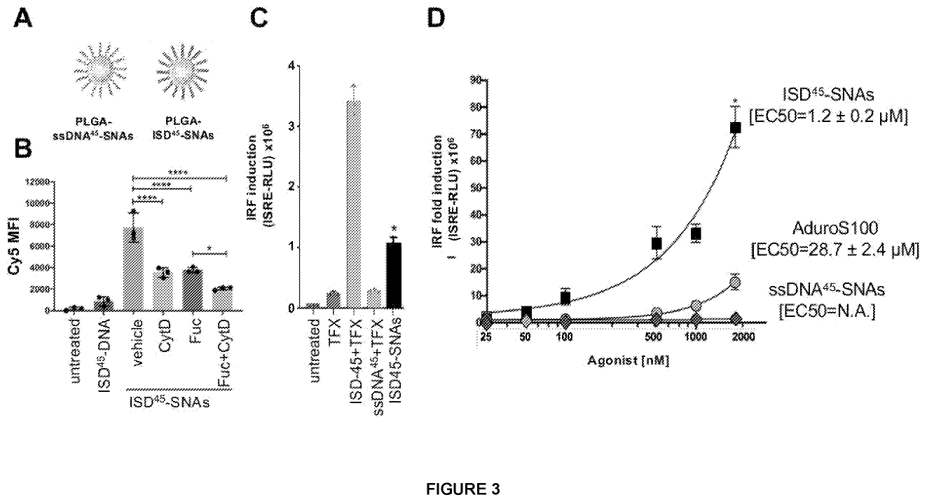

To exploit the SNA architecture for cGAS activation, we synthesized ISD45-SNAs by conjugating 13 nm gold nanoparticles with 45 base pair (bp) dsISD oligonucleotides (ISD⁴⁵-SNAs; FIG. 1A), as 45 bp ISD oligonucleotides represent the most used and widely characterized cGAS activator⁷. The terms “dsISD⁴⁵” and “ISD⁴⁵” are interchangeable as used herein. Citrate-stabilized gold nanoparticles were prepared using the Frens method^{4,8,9}. Thiolated dsDNA oligonucleotides were synthesized, purified using reverse-phase high performance liquid chromatography, and covalently linked to gold nanoparticle surfaces by sodium chloride salt aging^{10,11}. Electron microscopy and dynamic light scattering verified SNA^{ISD-45} shape and size (13 nm gold core, 72 nm diameter with DNA corona). In addition, by generating melting curves, using an OliGreen fluorescence assay, and determining the zeta potential, oligonucleotide incorporation was quantified^{4,8,9}. To assess direct engagement of the cGAS DNA sensing enzyme by SNAs^{ISD}, a cell-free cGAS activation assay was developed using human recombinant cGAS enzyme (FIG. 1B). A mixture of recombinant human cGAS enzyme, MgCl₂, ATP, and GTP was incubated with either ssDNA⁴⁵, ISD⁴⁵, cGAMP, ssDNA⁴⁵-SNAs, or ISD⁴⁵-SNAs. Upon cGAS enzyme deactivation, the mixture was added to cGAS-deficient RAW-Lucia™ macrophages, carrying stable integration of an IRF-inducible Lucia luciferase reporter to quantify IRF induction luminometrically. This macrophage line cannot synthesize cGAMP due to cGAS deficiency. As shown in FIG. 1C, ISD⁴⁵-SNAs, but not ssDNA⁴⁵-SNA-treated macrophages activated IRF to similar levels compared to free ISD⁴⁵ oligonucleotides or cGAMP-transfected cultures. Reflecting robust cell uptake, in particular in macrophages (FIG. 2A), ISD⁴⁵-SNAs potently activated IRFs when compared to free oligonucleotides, and to similar levels achieved with free oligonucleotides delivered via lipoplex (Lipofectamine 3000) transfection (TFX), in both RAW-Lucia™ macrophages and THP-1 cells, a human monocytic cell line. (FIGS. 2B-C). Dependency of IRF induction on functional cGAS-STING pathway components was subsequently assessed in isogenic cGAS-, STING-, and IRF-deficient macrophages (FIG. 2D), demonstrating that in the absence of STING, cGAS or IRF, IRF induction in response to SNA^{ISD-45} or ISD-45 treatment was blunted. In addition, multiplex cytokine profiling using ELISA and antibody-arrays revealed robust activation of NF-κB- and STAT6-induced pro-inflammatory cytokines, including IL-6, TNF-α and CCL2 in response to SNA^{ISD} treatment (FIGS. 2E-F). Importantly, as has been reported for gene-regulatory and TLR9-activating SNAs, the cGAS-activating properties of ISD⁴⁵-SNAs stem from the densely functionalized and highly oriented nucleic acid shell, and not from the nanoparticle core¹²⁻¹⁴. ISD⁴⁵-SNAs with 80 nm PLGA-based cores (FIG. 3A), similarly to 13 nm gold-based architectures (FIGS. 1 and 2), enter macrophages via phagocytosis and scavenger receptor-dependent endocytosis (FIG. 3B), and unlike ssDNA45-SNAs, promote IRF activation (FIG. 3C), and show higher EC₅₀ values than AduroS100, a CDN currently in clinical trials for non-CNS solid cancers (FIG. 3D).

### ISD⁴⁵-SNAs Induced M1 Macrophage Polarization Reduced Tumor Cell Viability and Antagonized Tumor Progression In Vivo

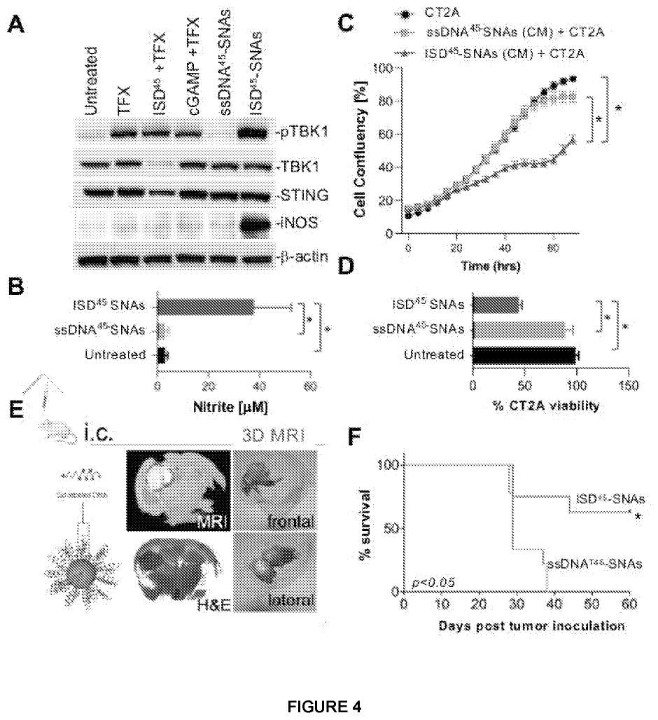

In addition to eliciting adaptive T cell responses against tumor, several studies indicated that activation of the cGAS-STING pathway antagonizes tumor growth by promoting the activation of pro-inflammatory M1-polarized macrophages. Since GBM are largely comprised of tumor-associated macrophages with anti-inflammatory M0/2-like phenotype, it was examined whether ISD45-SNAs can re-educate M0 macrophages to the M1 phenotype. In RAW-Blue™ macrophages, ISD45-SNAs, but not ssDNA⁴⁵-SNAs treatment triggered robust TBK1 phosphorylation, as well induction of cytokine-inducible nitric oxide synthase (iNOS), a classical marker of the proinflammatory macrophage state (FIG. 4A). Correspondingly, SNAs^{ISD-45} increased nitrite level in macrophage supernatants (FIG. 4B), and tumor cells treated with conditioned medium from SNA^{ISD-45}-exposed macrophages or co-cultured with SNA^{ISD-45}-exposed macrophages reduced tumor cell proliferation and viability (FIGS. 4C-D). When injected intratumorally, SNAs pervasively infiltrated intracranial tumors, as demonstrated by MR imaging of SNAs functionalized with Gd(III)-labeled DNA oligonucleotides (FIG. 4E). To investigate whether cGAS-agonistic SNAs reduce GBM tumor progression in vivo, syngeneic luciferase-modified CT2A orthotopic engraftment models received a single intratumoral dose of either ssDNA⁴⁵- or ISD45-SNAs (corresponding to 0.15 mg/kg of dsDNA) 7 days post tumor cell inoculation. Analysis of animal subject survival using the Kaplan-Meyer method revealed that ISD45-SNAs, but not ssDNA⁴⁵-control SNAs significantly slowed down GBM tumor progression and promoted long-term survival in more than 50% of treated female animal subjects (FIG. 4F).

### Non-Invasive Nose-to-Brain Delivery of ISD45-SNAs

Localized ISD45-SNA delivery into the tumor bed circumvents the blood-brain barrier, avoids toxicity due to systemic exposure, and therefore, remains an attractive and translatable option for GBM treatment. Therefore, intratumoral administration of cGAS-agonistic SNAs is explored and optimized, as described below. In addition to direct intratumoral administration of cGAS-agonistic SNAs, non-invasive SNA nose-to-brain delivery is explored, leveraging the increased stability and bioavailability of SNA-formulated oligonucleotides and their propensity to pervasively infiltrate tumor parenchyma. Similar to intratumoral delivery, intranasal delivery bypasses the blood-brain barrier and minimizes systemic exposure. Furthermore, the advent of new intranasal delivery technologies together with recent preclinical and clinical studies demonstrating safety and effectiveness of nose-to-brain delivery for the delivery of a broad spectrum of therapeutics, including perillyl alcohol (NEO100) for the treatment of recurrent GBM [currently being evaluated in a phase I/II clinical trial], credential intranasal delivery as a promising and non-invasive delivery method for GBM treatment, and motivate studies outlined in the grant application, to comprehensively evaluate and optimize SNA nose-to-brain delivery.

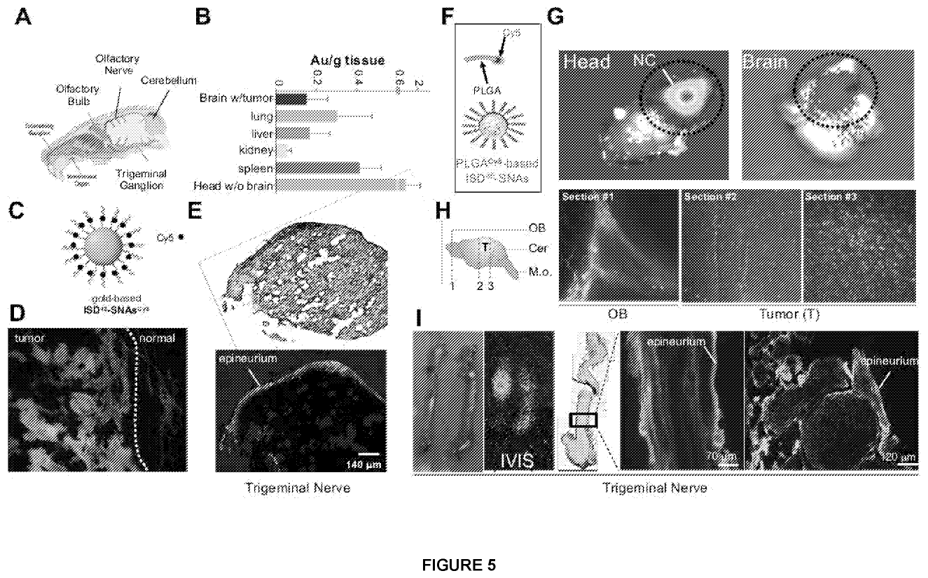

Intranasally administered drugs reach the CNS through pathways that involve olfactory and trigeminal nerves that connect the nasal passage to the CNS (FIG. 5A). ICP-MS analysis of Au content demonstrated that approximately 10% of the total ISD45-SNA dosage administered accumulated in tumor-bearing brain when administered intranasally (FIG. 5B). Similarly, using Cy5-labeled gold-based ISD45-SNAs (FIG. 5C), fluorescence microscopy imaging revealed SNA accumulation in tumor elements (FIG. 5D) and within the epineurium of the trigeminal nerve (FIG. 5E). Similarly, Cy5-labeled PLGA-based SNAs (FIG. 5F) showed brain accumulation, with Cy5-labeled structures detectable in the both the olfactory bulb and within the intracranial tumor (FIG. 5G-H). ISD45-SNAs were also found within the epineurium, perineurium, and, to a lesser extent, the endoneurium of the trigeminal nerve (FIG. 51). These data suggested that ISD45-SNA nose-to-brain delivery occurred at least in part along extracellular components of the trigeminal nerve.

### Intranasal Administration of ISD45-SNAs Reduces GBM Progression in a STING-Dependent Manner and Promotes Long-Term Survival in Combination with Immune-Mediated Checkpoint Inhibition

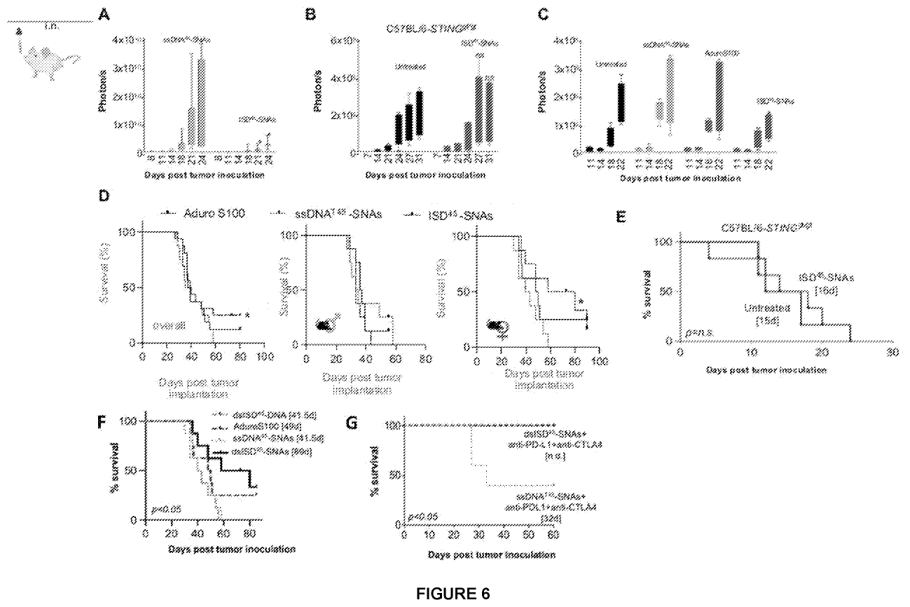

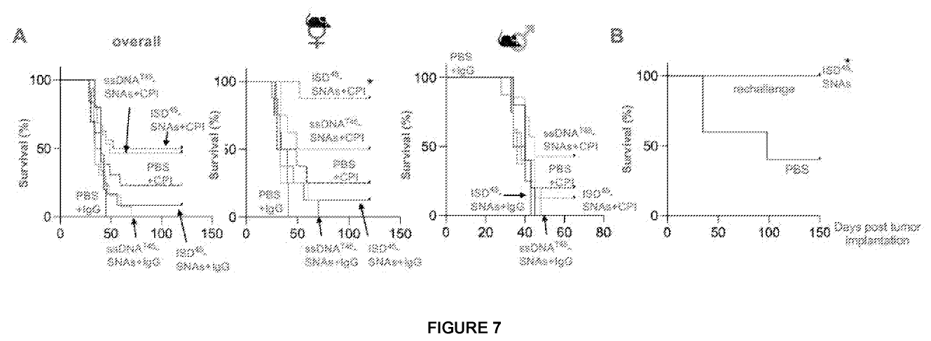

We implanted luciferase-labeled CT2A tumor cells into either C57BL/6-wt or C57BL/6-STING goldenticket (gt/gt) syngeneic hosts. ISD45-SNAs delivered via a single intranasal administration reduced tumor progression and increased animal survival in CT2A-bearing C57BL/6 wild-type mice (FIG. 6A) and did not impact tumor progression in STING-deficient gt/gt mice (FIG. 6B). Therapeutic SNA benefit was significantly higher compared to ADU-S100, a cyclic dinucleotide currently in clinical testing (FIG. 6C). Kaplan-Meyer survival analyses revealed that ISD45-SNAs extended animal subject survival in C57BL/6 wild-type, in particular in female but not in male animal subjects (FIG. 6D), and failed to extend survival in C57BL/6 hosts lacking functional STING in the tumor microenvironment, when compared to untreated or ssDNA45-SNA-treated mice (FIGS. 6E-F). When co-treated with ISD45-SNAs and immune-mediated checkpoint inhibition (anti-PD1+ anti-CTLA4), one-time intranasal delivery abolished GBM tumor development in almost all female animals (FIG. 6G), but had no effect on male mice (FIG. 7A). Rechallenge of long-term surviving female mice with tumor cells injected into the contralateral hemisphere resulted in long-term survival in all mice, indicative of long-term anti-tumor memory (FIG. 7B).

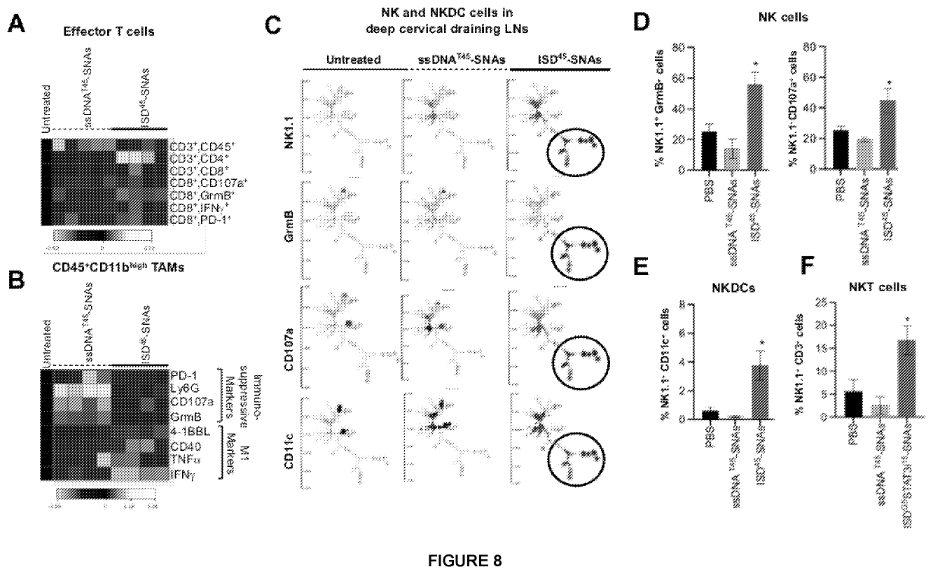

Analysis of tumor-associated and peripheral immune system composition in mice injected with SNAs through direct intratumoral administration revealed enrichment of effector T cells (FIG. 8A), M1-polarized macrophages (FIG. 8B), as well as activated NK cell populations in ISD45-SNAs-treated mice in comparison to untreated ssDNA⁴⁵-SNA-treated animal subjects (FIGS. 8C-F). These data demonstrated that the anti-tumor activity of ISD45-SNAs was greater than anti-tumor effect of CDNs, when consideration is given to non-invasive nose-to-brain delivery, required functional STING in the tumor microenvironment, and could be boosted by combination with immune checkpoint inhibition selectively in female but not in male animal subjects.

### Example 2

### Development of Bimodal ISD45/STAT3i-SNAs

Signal Transducer and Activator of Transcription 3 (STAT3) is as a critical transcriptional regulator of innate and adaptive immunity, and actionable immune target¹⁵. Recent studies identified STAT3 inhibitors as a novel class of immunotherapeutics that can enhance anti-tumor immunity in response to STING pathway activation¹⁵. Due to the absence of strict sequence and structural parameters that guide the engagement of dsDNA with cGAS, we examined SNA structures formulated with oligonucleotides that in addition to cGAS activation can function as STAT3 decoy oligonucleotides, sequestering STAT3 in the cytosol and preventing it from translocating to and activating transcription in the cell nucleus. Recent studies defining the interaction of ISD oligonucleotides with cGAS suggested that a minimum length of 15 bp is required to promote the synthesis of cGAMP1, and identified palindromic sequences flanked by unpaired guanosine trimers (G3) that enhanced the immunostimulatory effect of otherwise inactive blunt sequences in a cGAS-STING dependent manner.

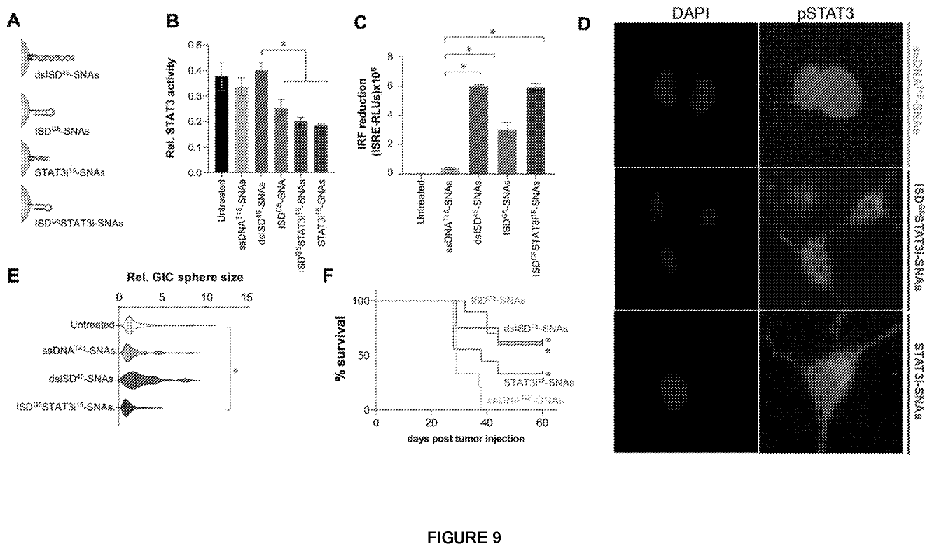

To increase anti-tumor immune responses of prototypic ISD45-SNAs, we designed SNAs conjugated with a 15 bp ISD oligonucleotide derived from STAT3 decoy sequences (ISD15STAT3i-SNAs) and a hairpin structure that contains a G5 repeat element flanked by STAT3 decoy palindromic sequences (ISD^{G5}STAT3i-SNAs; FIG. 9A). Modified gel shift assay and Hill-Langmuir computing showed that all SNAs formed a complex with cGAS with KD ranging from 0.17 to 0.24 nM (FIG. 9B, C), and that both ISD15STAT3i and ISD^{G5}STAT3i-SNAs, but not ISD45-SNAs, directly bound STAT3 with KD of 0.045 and 0.07 nM (FIG. 9D, E). Consistent with cell-free binding profiles, all SNA architectures induced IRF3 activation, as assessed in both cell-free and cell-based IRF3 reporter assays (FIG. 9F, G), but only ISD15STAT3i and ISD^{G5}STAT3i-SNAs, but not ISD45-SNAs, repressed IL-6 level as assessed in HEK-Blue IL6 reporter cells (FIG. 9H).

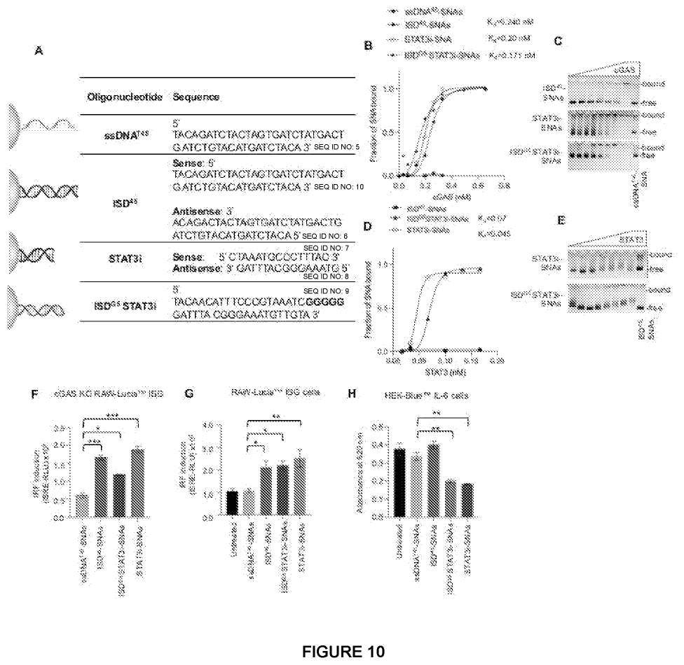

The therapeutic effect of bimodal ISD¹⁵STAT3i and ISD^{G5}STAT3i-—SNAs compared to monofunctional ISD45-SNAs in CT2A- and advanced syngeneic orthotopic glioblastoma models using tumor neurospheres derived from a genetically engineered glioblastoma mouse model was evaluated. In this model, glioma formation is driven by tamoxifen-inducible CNS-specific deletion of Quaking, a STAR family RNA-binding protein that regulates multi-potency and self-renewal in neural and glioma stem cells, and p53 and PTEN tumor suppressors (genotype: Nestin-CreER^{T2}; Qki^{L/L}; Pten^{L/L}; Trp53^{L/L}, referred to as QPP model). Tumor neurospheres isolated from QPP tumors and orthotopically implanted into syngeneic hosts resulted in glioblastoma tumor formation with a latency of 40-70 days. Tumor neurospheres cultures derived from different QPP tumors showed a spectrum of sensitivity to checkpoint inhibition. QPP4 tumors were resistant to checkpoint inhibitor treatment, while explant tumor models derived from QPP7 cells were sensitive to anti-CTLA4 and showed a trend toward sensitivity for anti-PD1 blockade. In checkpoint inhibitor-sensitive CT2A and QPP7 models, ISD45-SNAs and ISD15STAT3i-SNAs, but not ISD^{G5}STAT3i-SNAs, potently antagonized tumor progression and prolonged survival in female, but not in male animal subjects, with 60-80% of mice showing long-term survival (FIG. 10A, B). In the checkpoint blockade-resistant QPP4 model, using female mice only, treatment with ISD^{G5}STAT3i, but not ISD45-SNAs and ISD¹⁵STAT3i-SNAs, resulted in significant albeit lessened benefit, with 30% of mice showing long-term survival (FIG. 10C).

### Immune-Mediated Changes in Response to SNA Treatment are Sex-Specific

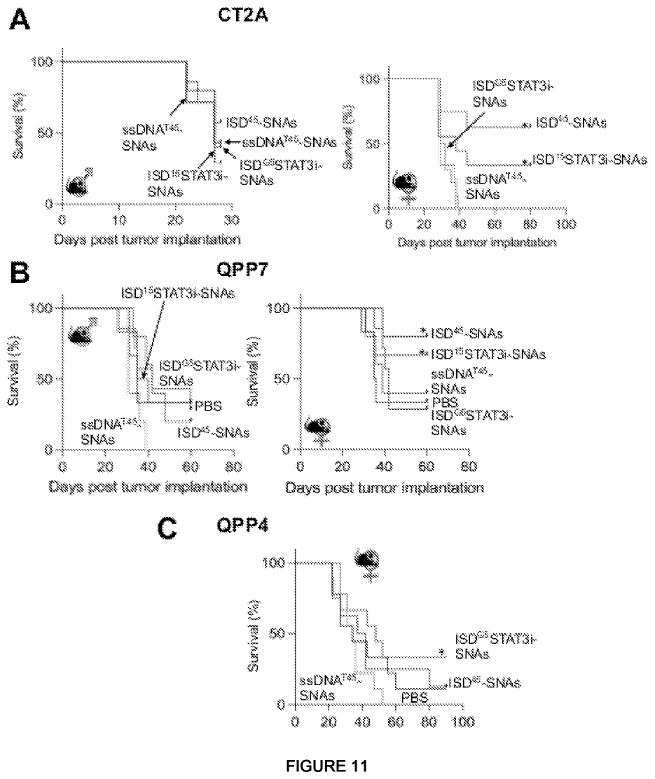

To define immune-mediated changes in SNA-versus control-treated male and female animal subjects, gene expression analysis of the TME was performed using the Nanostring PanCancer Immune profiling panel. QPP4 tumor-bearing C57BL/6 mice were treated with PBS, ssDNA45-SNA, ISD45-SNAs, ISD¹⁵STAT3i-SNAs or ISD^{G5}STAT3i-SNAs via intracranial administration on day 8 post tumor cell inoculation, and RNA was isolated from tumor-infiltrating immune cells 30 days post tumor implantation. In females, unsupervised clustering of z-score-ranked gene expression revealed clear separation between treatment and control groups (FIG. 11A), with SNA treatment inducing immune-activating and downregulating immune-suppressive genes. Upregulated genes include inducers of interferons (Tbk1, Irf3, Ifnb1, Ifna1), M1 polarization (Mst1r, Mx1), T cell co-stimulation (Icam, Ncam, Icosl, CD28), T cell activation (CD69), and immune cell migration (Cc/3, Cc/4, Cxc/12, Csf1). In addition, immune-suppressive genes expressed by activated M2 macrophages and myeloid cells (Arg1, Arg2, Msr1, IL-10, CC/17, CC122) and genes responsible for suppression of CD4+ and CD8+ effector T cell functions (Pdcd1, FoxP3, Vegfa, Hifla) were significantly downregulated with SNA treatment compared to PBS and ssDNA45-SNA controls. In stark contrast, SNA treatment in male mice downregulated a wide array of genes, including immune checkpoint receptors (Ctla4, Havcr2, Icos), and genes required for the activation of innate immunity (Isg20, Isg15, Cxcl10), and T cells (CD28, CD3, Grmb, CD8) (FIG. 11B). Nanostring gene-set pathway analysis revealed that in the female QPP4 glioma model, ISD^{G5}-STAT3i-SNAs upregulated 25/30 immune cell pathways implicated in macrophage, microglia, dendritic cell, T-cell and leukocyte functions, inflammation, as well as chemokines and cytokines and responses to pathogens, indicative of more potent pro-inflammatory and anti-tumor activity of ISD^{G5}STAT3i-SNAs, when compared to ISD45-SNAs, and ISD¹⁵STAT3i SNAs (FIG. 11C). In male QPP4, ISD^{G5}-STAT3i-SNAs and ISD45-SNAs failed to elicit any immune response and downregulated (30/30) all the innate and adaptive immune mechanisms (FIG. 11D).

To characterize sexually dimorphic genes signatures, in particular induced in response to ISD^{G5}-STAT3i-SNAs, we performed unsupervised clustering for all female (n=3) and male (n=4) samples (FIG. 11E-G), followed by gene ontology pathway analysis. As shown in FIG. 11F, ISD^{G5}STAT3i-SNAs treatment resulted in a 15-fold enrichment for adaptive immune response.

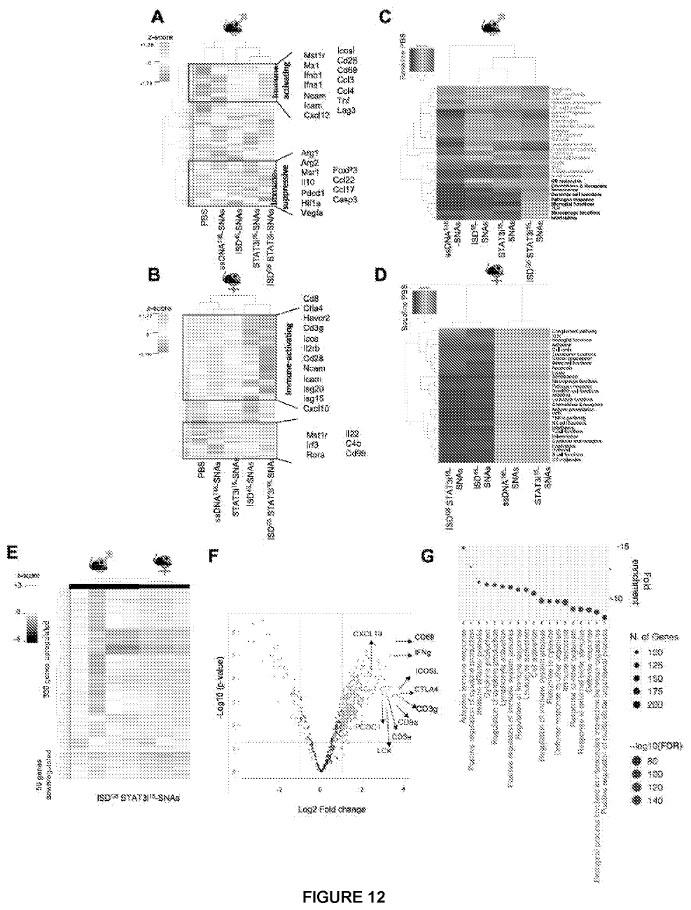

To begin to analyze the molecular basis for sexual dimorphic responses to SNA^{ISD/STAT3i} treatment, cGAS protein expression of total spleen (FIG. 12A), BMDMs (FIG. 12B) and TAMCs isolated from tumor-bearing mice treated with either PBS, ssDNA45-SNAs or ISD45-SNAs (FIG. 12C) was analyzed using Western Blot and flow cytometry analysis. Results indicated higher cGAS expression in female versus male immune cells. Thus, these data suggest cGAS expression level as a putative predictive biomarker for cGAS-agonistic SNA responses.

### DISCUSSION

Stimulation of the cGAS-STING pathway increases T cell activation and tracking into the tumor and reverses the immunosuppressive phenotype of myeloid cells. Direct targeting of the STING receptor using synthetic cyclic dinucleotide (CDN) ligands represents an attractive immunotherapeutic strategy for the treatment of lymphocyte-depleted and myeloid cell-enriched tumors, such as Glioblastoma (GBM). This approach, however, is limited by the metabolic instability of CDNs, and the presence of immune evasive and STING-inhibitory mechanisms in the tumor microenvironment (TME), including and especially those orchestrated by the master transcriptional regulator of inflammation and immunity STAT3 (Signal Transducer and Activator of Transcription 3). To activate cGAS anti-tumor responses and concomitantly inhibit STAT3-driven immunosuppression in the GBM TME, Spherical Nucleic Acids (SNAs) that consist of a nanoparticle core densely functionalized with a shell of radially oriented DNA oligonucleotides containing STAT3 decoy sequence were generated. Bimodal SNA architectures, via multivalent and high-affinity binding of cGAS and STAT3 proteins, induced type I interferon (IFN), prevented STAT3 nuclear translocation and transactivation; reduced tumor cell viability; promoted immune activation in the GBM tumor microenvironment, and elicited strong anti-tumor activity. Striking sexual dimorphic responses to SNA treatment was observed, with curative effects of bimodal SNAs in female, but not in male animal subjects. These studies established multifunctional SNAs as a first-in-class, single-entity nucleic acid therapeutic for targeting multiple immune pathways in GBM.

Since DNA recognition by cGAS is sequence-independent, it was tested whether replacing the canonical cGAS agonistic oligonucleotide sequence with palindromic sequences known to bind to and sequester STAT3, would yield SNA architectures capable of promoting cGAS-STING pathway activation and concurrently inhibiting STAT3 function (FIG. 9A). Indeed, ISD^{G5} and ISD^{G5/STAT3i}-SNAs blocked STAT3 transactivation, as assessed by a STAT3 reporter assay (FIG. 9B) and promoted IRF activation (FIG. 9C). Consistent with reduced STAT3 transactivational activity in response to SNA treatment, bimodal SNAs sequestered pSTAT3 in the cytoplasm (FIG. 9D), reduced sphere size of glioma stem cell cultures (FIG. 9E), and prolonged animal subject survival when given via intracranial administration (FIG. 9F).

### Materials and Methods

Cell lines. Raw-Lucia™ ISG, Raw-Lucia™ ISG cGAS KO, and HEK-blue STAT3 reporter cell lines were purchased from InvivoGen (San Diego, CA, USA) and were cultured according to manufacturer's protocols. QPP4, QPP7 cell lines were generated and gifted by Dr. Amy Heimberger's lab. QPP tumor neurospheres were isolated from GBM tumors arising in a genetically engineered mouse model (genotype: Nestin-Cre-ERT2; Quaking^{L/L}; p53^{L/L}; PTEN^{L/L}). Cells were maintained in NeuroCult Proliferation kit (media and supplement, Stem Cell) supplemented with EGF (20 μL/10 ml medium) and FGF (10 μL/10 ml medium). GL261-luc, GL261, and CT2A cell lines were maintained in Dulbecco's Modified Eagle's Medium (DMEM, Fisher Scientific) supplemented with 10% fetal bovine serum (FBS, Hyclone). All media were supplemented with 1% penicillin/streptomycin (Invitrogen) and cell lines cultured at 37° C. in a humidified environment with 5% CO2. All cell lines tested negative for mycoplasma either in house using the MycoAlert Plus Mycoplasma detection kit (Lonza) or by Charles River Research Animal Diagnostic Services (CR RADS, MA USA).

Murine tumor models for in vivo SNA testing. All animals were used under an approved animal study protocol, were maintained at the animal facilities of Northwestern University, and treated in accordance with the regulations and guidelines of the Institutional Animal Care and Use Committee (IACUC). Female or male C57BL/6J mice (The Jackson Laboratory) aged 6-8 weeks were implanted intracranially with CT2A (8×10⁴ cells/mouse), QPP7 or QPP4 cells (100×10⁴ cells/mouse). Each mouse was anesthetized using intraperitoneal (i.p.) injection of ketamine/xylazine and the surgical area was cleaned with alcohol and Betadine. After an incision was made in the scalp, a 0.7 mm burr hole was created, using a microsurgical drill, 2 mm to the lateral right of the sagittal suture and 4 mm behind the coronal suture. The cell lines loaded in a 25-μL Hamilton syringe were implanted using a stereotactic frame (Stoelting). After surgery, the skin was closed with sutures. Mice were randomly assigned to control or treatment groups (n=6-10/group) after tumor implantation. Upon reaching an average size of approximately 100 mm³, the tumors were treated via intratumoral or intranasal injections of either PBS, ssDNA^{T45}-SNA, ISD45-SNAs, STAT3i¹⁵-SNAs, ISD^{G5}STAT3i-SNAs nanoparticle formulations. Mice were sacrificed upon observation of neurological impairment (lethargy, failure to ambulate, lack of feeding, or loss of >20% body weight) that typically occurred 48 hours before death. Survival analysis was performed using the Kaplan-Meier method, and statistical significance was assessed using the log-rank (Mantel-Cox) test.

Intranasal delivery. The therapeutic efficacy of ISD45-SNA was also evaluated upon intranasal (i.n.) administration in CT2A (8×10⁴ cells/mouse) mouse models. One dose of sterile saline or SNA (15 μmol by Au per kg weight of mouse) was given on the 7th day after intracranial tumor cells implantation. 2 μl fractions of saline or SNAs were given in each nostril every 5 min.

Cannula implantation and intracranial SNA injection. Mice were anesthetized with ketamine/xylazine as above described. A 26-gauge sterile plastic guide cannula was installed into the skull at a depth of 2 mm through a burr hole. The implanted cannula was secured, and the skin was closed, by applying surgical glue. Standard post-surgery care was given according to the IACUC-approved protocol. During injection, a 33-gauge sterile hamilton syringe, equipped with a sleeve designed to extend 1 mm beyond the tip of the guide cannula, was used to implant cells or inject therapeutics diluted in sterile 0.9% saline. After injection, the cannula was covered with a 33-gauge dummy cannula or cap.

NanoString nCounter analysis. 1×10⁶ QPP4 cells were implanted intracranially into 6-8 weeks old male and female C57BL/6 mice via a cannula as described above. The mice were intracranially treated on day 7th post tumor cell injection with sterile PBS, ssDNA^{T45}-SNAs, ISD⁴⁵-SNAs, or ISD^{G5}STAT3i-SNAs (n=4 per group). The dose of the AuNPs was 15 μmol by Au per kg. On day 30 post SNA treatment, the mice were euthanized and perfused with 15 ml of DPBS intracardially. The perfused brain tissue was further processed with the “mouse brain tumor dissociation kit” (Miltenyi Biotec, #130-096-730) to obtain isolated single cell tumor cells and tumor-infiltrating immune cell suspensions. The immune cells were separated from the rest of the brain tissue by 30/70 Percoll gradient separation (GE Healthcare).

For the Nanostring analysis, total RNA was extracted using the kit (Invitrogen™ PureLink™ RNA Mini Kit) according to the manufacturer's protocol. Quality control of the isolated RNA was performed using an Agilent bioanalyzer and NanoDrop 8000 (Thermo Scientific). RNA isolated from immune cells was analyzed by NanoString nCounter using the PanCancer Immune Profiling Panel. Briefly, 150 ng of RNA was hybridized for 16 hours using capture and reporter probes. The samples were then immobilized into a cartridge using the nCounter™ prep-station. Digital images were processed within the nCounter™ Digital Analyzer instrument, with 555 fields of view (fov) being collected for each sample. The reporter probe counts (i.e., the number of times the color-coded barcode for that gene is detected) were analyzed using the nSolver™ package (version 3.0), Advanced nSolver Analysis module (version 1.0.36) and ROSALIND software. The QC, normalization, differential expression, and pathway analysis were performed using the nSolver advance analysis module and ROSALIND software according to the guidance given by manufacturers. The fold change was calculated by comparing against the average normalized gene expression values of PBS-treated tumors.

### In Vitro Experiments to Assess cGAS and STAT3 Engagement

Electrophoretic mobility shift assay. Recombinant human cGAS protein (Cayman) or recombinant human STAT3 protein were incubated with SNAs at room temperature for 1 hour in buffer (20 mM Tris buffer pH 7.2; sample volume of 50 ul). The reaction contained 15 umoles of Au and cGAS/STAT3 proteins (0.1 ug to 2 ug). Reactions were analyzed by electrophoresis on a 1% agarose gel in 1× TAE buffer. The image of the gels was scanned using Biorad gel doc system using FAST blast selection and analyzed for Au band intensity using FIJI.

IRF3 Luciferase Reporter Assay. To measure IRF3 activation, Raw-Lucia™ ISG (InvivoGen) cells were seeded in a 96 well plate at a density of 3.0×10⁴ cells/well. The next day, cells were treated with SNAs or PBS at concentration of 15 umoles of Au. After incubation for 24 hours, supernatants were transferred to a new white clear bottom 96 well plate. Then luciferase luminescence was recorded with a Synergy H2 Microplate Reader (BioTek) using QUANTI-Luc Luciferase reagent (Invivogen) according to manufacturer's suggested protocol.

IL-6 reporter assay. To measure STAT3 activation, HEK-Blue™ IL-6 cells were seeded in a 96 well plate at a density of 3.0×10⁴ cells/well and were cultured overnight. The next day, cells were incubated with SNAs or PBS at concentration of 15 umoles of Au for 4 hours followed by 12 hours of IL-6 (1.5 ng/ml) treatment. Upon IL-6 stimulation, HEK-Blue™ IL-6 cells trigger the activation of STAT3 and the subsequent secretion of SEAP. Levels of STAT3-induced SEAP were monitored using QUANTI-Blue™ Solution.

cGAS cell-free activity assay. For assessment of cGAS activity in a cell-free assay, 60 nM of recombinant human cGAS protein (Cayman) was mixed with ATP (5 mM), GTP (300 μM) and SNAs (15 umoles of Au) in reaction buffer (20 mM Tris-HCl, pH 7.5, 1.5 mM MgCl2). After incubation at 37° C. for 2 hours, the reaction was terminated by heating the reaction mixture at 95° C. for 5 min to denature proteins, which were removed by centrifugation at 20,000×g for 5 min. The supernatant was transfected into Raw-Lucia™ ISG cGAS KO Raw Lucia Blue macrophages (InvivoGen). The cells were cultured at 37° C. for 24 hours and then luminescence was recorded with a Synergy H2 Microplate Reader (BioTek) using QUANTI-Luc Luciferase reagent (Invivogen) according to manufacturer's suggested protocol.

Statistical analysis. All statistical analyses were performed using GraphPad Prism software, version 9.0. Non-parametric unpaired Student t-test and Mann-Whitney U-test were used to assess the significance of differences between two groups. All the data were reported as mean±SEM. Multiple groups were analyzed with one-way ANOVA along with by Tukey's multiple comparison post hoc test. Kaplan-Meier plots were generated to determine relative survival of glioma bearing mice under different courses of treatment. The p values for curve comparisons were calculated using the Log-rank method followed by Bonferroni correction.

cGAS expression analysis. CT-2A bearing mice treated with PBS, ssDNA45-SNAs, or ISD45-SNAs were euthanized on day 15^{th} by CO² and perfused with 5 ml of DPBS intracardially. Brain/tumor single cell suspension was obtained in Hank's balanced salt solution (HBSS, Gibco) using a tissue homogenizer (Potter-Elvehjem PTFE pestle), followed by removal of myelin and debris by 30/70 Percoll gradient separation (GE Healthcare). Glioma infiltrating immune cells were collected into complete RPMI for phenotyping or ex vivo study, and stained for cGAS expression. For this intracellular staining, cells were treated with STIM cocktail (BD Biosciences) for 4 hrs prior to staining. Cells were washed, stained with Zombie NIR live/dead viability dye (ThermoFisher Scientific) in PBS, washed two additional times, and stained with a cocktail of surface antibodies for CD11 b and CD45 diluted to 1:200 (final concentration) in PBS at 4° C. for 30 min. For intracellular staining of cGAS, cells were fixed and permeabilized overnight in permeabilization buffer (eBioscience), incubated with cGAS anti-rabbit primary antibody for 1 hr at RT and then stained with a goat anti-rabbit AF488 (Thermofisher A-11008) at 1:2000 concentration for 30 min. The samples were washed, acquired on a Cytek Aurora (Northern Lights), and analyzed using Cytobank V7.3.0.

For cGAS western blot analysis, total homogenized spleen or murine BMDMs were lysed in RIPA buffer to generate whole cell lysates, and subjected to Western Blot analysis for cGAS and β-actin (loading control).

Sequences disclosed herein:

| SEQ | |
|---|---|
| ID NO | Sequence |
| 1 | 5′-CATTTCCCGTAAATC-3′ |
| 2 | 5′-GATTTACGGGAAATG-3′ |
| 3 | 5′-ACATCTAGTACATGTCTAGTCAGTATCTAGTGATCATCTA |
| | GACAT-3′ |
| 4 | 5′-ATGTCTAGATGATCACTAGATACTGACTAGACATGTACTA |
| | GATGT-3′ |
| 5 | 5′-TACAGATCTACTAGTGATCTATGACTGATCTGTACATGAT |
| | CTACA-3′ |
| 6 | 5′-ACATCTAGTACATGTCTAGTCAGTATCTAGTGATCATCAG |
| | ACA-3′ |
| 7 | 5′-CTAAATGCCCTTTAC-3′ |
| 8 | 5′-GTAAAGGGCATTTAG-3′ |
| 9 | 5′-TACAACATTTCCCGTAAATCGGGGGGATTTACGGGAAATG |
| | TTGTA-3′ |
| 10 | 5′-TACAGATCTACTAGTGATCTATGACTGATCTGTACATGAT |
| | CTACA-3′ |

### REFERENCES

1 Corrales, L., McWhirter, S. M., Dubensky, T. W., Jr. & Gajewski, T. F. The host STING pathway at the interface of cancer and immunity. The Journal of clinical investigation 126, 2404-2411, doi:10.1172/JC186892 (2016). 4922692
2 Ohkuri, T., Ghosh, A., Kosaka, A., Sarkar, S. N. & Okada, H. Protective role of STING against gliomagenesis: Rational use of STING agonist in anti-glioma immunotherapy. Oncoimmunology 4, e999523, doi:10.1080/2162402λ.2014.999523 (2015). 4485761
3 Mirkin, C. A. & Stegh, A. H. Spherical nucleic acids for precision medicine. Oncotarget 5, 9-10 (2014). 3960185
4 Jensen, S. A. et al. Spherical Nucleic Acid Nanoparticle Conjugates as an RNAi-Based Therapy for Glioblastoma. Science translational medicine 5, 209ra152, doi:10.1126/scitranslmed. 3006839 (2013)
5 Radovic-Moreno, A. F. et al. Immunomodulatory spherical nucleic acids. Proceedings of the National Academy of Sciences of the United States of America 112, 3892-3897, doi:10.1073/pnas. 1502850112 (2015).4386353
6 Skakuj, K. et al. Conjugation Chemistry-Dependent T-Cell Activation with Spherical Nucleic Acids. Journal of the American Chemical Society 140, 1227-1230, doi:10.1021/jacs.7b12579 (2018).5831183
7 Stetson, D. B. & Medzhitov, R. Recognition of cytosolic DNA activates an IRF3-dependent innate immune response. Immunity 24, 93-103, doi:10.1016/j.immuni.2005.12.003 (2006)
8 Giljohann, D. A., Seferos, D. S., Prigodich, A. E., Patel, P. C. & Mirkin, C. A. Gene regulation with polyvalent siRNA-nanoparticle conjugates. Journal of the American Chemical Society 131, 2072-2073, doi:10.1021/ja808719p (2009).2843496
9 Kouri, F. M. et al. miR-182 integrates apoptosis, growth, and differentiation programs in glioblastoma. Genes & development 29, 732-745, doi:10.1101/gad.257394.114 (2015).4387715
10 Mirkin, C. A., Letsinger, R. L., Mucic, R. C. & Storhoff, J. J. A DNA-based method for rationally assembling nanoparticles into macroscopic materials. Nature 382, 607-609, doi:10.1038/382607a0 (1996)
11 Rosi, N. L. et al. Oligonucleotide-modified gold nanoparticles for intracellular gene regulation. Science 312, 1027-1030, doi:10.1126/science.1125559 (2006)
12 Calabrese, C. M. et al. Biocompatible infinite-coordination-polymer nanoparticle-nucleic-acid conjugates for antisense gene regulation. Angewandte Chemie 54, 476-480, doi:10.1002/anie.201407946 (2015). PMC4314394
13 Cutler, J. I. et al. Polyvalent nucleic acid nanostructures. Journal of the American Chemical Society 133, 9254-9257, doi:10.1021/ja203375n (2011). PMC3154250
14 Morris, W., Briley, W. E., Auyeung, E., Cabezas, M. D. & Mirkin, C. A. Nucleic acid-metal organic framework (MOF) nanoparticle conjugates. Journal of the American Chemical Society 136, 7261-7264, doi:10.1021/ja503215w (2014)
15 Tripathi, S., Najem, H., Mahajan, A. S., Zhang, P., Low, J. T., Stegh, A. H., Curran, M. A., Ashley, D. M., James, C. D., Heimberger, A. B. cGAS-STING pathway targeted therapies and their applications in the treatment of high-grade glioma. F1000Res. 11:1010 (2022).

What is claimed is:

1. A spherical nucleic acid (SNA) comprising:
(a) a nanoparticle core; and
(b) a shell of oligonucleotides attached to the external surface of the nanoparticle core, the shell of oligonucleotides comprising a double-stranded DNA oligonucleotide or single-stranded stem loop DNA oligonucleotide that activates cyclic GMP-AMP synthase (cGAS) and is at least 15 base pairs in length.

2. The SNA of claim 1, wherein the shell of oligonucleotides comprises a plurality of double-stranded DNA oligonucleotides and/or single-stranded stem loop DNA oligonucleotides, each of which activates cGAS and is at least 15 base pairs in length.

3. The SNA of claim 1, wherein the shell of oligonucleotides consists of a plurality of double-stranded DNA oligonucleotides and/or single-stranded stem loop DNA oligonucleotides, each of which activates cGAS and is at least 15 base pairs in length.

4. The SNA of any one of claims 1-3, wherein the shell of oligonucleotides comprises a plurality of double-stranded DNA oligonucleotides and each of the plurality of double-stranded DNA oligonucleotides comprises one strand comprising SEQ ID NO: 3 and another strand comprising SEQ ID NO: 4.

5. The SNA of any one of claims 1-3, wherein the shell of oligonucleotides consists of a plurality of double-stranded DNA oligonucleotides and each of the plurality of double-stranded DNA oligonucleotides comprises one strand comprising SEQ ID NO: 3 and another strand comprising SEQ ID NO: 4.

6. The SNA of any one of claims 1-5, wherein the double-stranded DNA oligonucleotide and/or single-stranded stem loop DNA oligonucleotide inactivates signal transducer and activator of transcription 3 (STAT3).

7. The SNA of any one of claims 1-6, wherein the shell of oligonucleotides comprises a plurality of double-stranded DNA oligonucleotides and/or single-stranded stem loop DNA oligonucleotides, each of which activates cGAS, inactivates signal transducer and activator of transcription 3 (STAT3), and is at least 15 base pairs in length.

8. The SNA of any one of claims 1-6, wherein the shell of oligonucleotides consists of a plurality of double-stranded DNA oligonucleotides and/or single-stranded stem loop DNA oligonucleotides, each of which activates cGAS, inactivates signal transducer and activator of transcription 3 (STAT3), and is at least 15 base pairs in length.

9. The SNA of any one of claims 1-8, wherein the nanoparticle core is a metallic core, a semiconductor core, an insulator core, an upconverting core, a micellar core, a dendrimer core, a liposomal core, a polymer core, a metal-organic framework core, a lipid nanoparticle core, a protein core, or a combination thereof.

10. The SNA of claim 9, wherein the polymer is polylactide, a polylactide-polyglycolide copolymer, a polycaprolactone, a polyacrylate, alginate, albumin, polypyrrole, polythiophene, polyaniline, polyethylenimine, poly(methyl methacrylate), poly(lactic-co-glycolic acid) (PLGA), or chitosan.

11. The SNA of claim 9, wherein the nanoparticle core comprises gold, silver, platinum, aluminum, palladium, copper, cobalt, indium, cadmium selenide, iron oxide, fullerene, metal-organic framework, silica, zinc sulfide, or nickel.

12. The SNA of claim 9, wherein the lipid nanoparticle core comprises an ionizable lipid, a phospholipid, a sterol, and a lipid-polyethylene glycol (lipid-PEG) conjugate.

13. The SNA of claim 12, wherein each oligonucleotide in the shell of oligonucleotides is covalently attached to the exterior of the lipid nanoparticle core through the lipid-PEG conjugate.

14. The SNA of claim 9, wherein the liposomal core comprises a plurality of lipid groups.

15. The SNA of claim 14, wherein the plurality of lipid groups comprises a lipid selected from the group consisting of the phosphatidylcholine, phosphatidylglycerol, and phosphatidylethanolamine families of lipids.

16. The SNA of claim 15, wherein the lipid is selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE).

17. The SNA of any one of claims 9-16, wherein at least one oligonucleotide in the shell of oligonucleotides is attached to the exterior of the liposomal or lipid nanoparticle core through a lipid anchor group.

18. The SNA of claim 17, wherein the lipid anchor group is attached to the 5′ end or the 3′ end of the at least one oligonucleotide.

19. The SNA of claim 17 or claim 18, wherein the lipid anchor group is tocopherol, DOPE lipid, or cholesterol.

20. The SNA of any one of claims 1-19, wherein the shell of oligonucleotides comprises one or more additional oligonucleotides.

21. The SNA of claim 20, wherein the one or more additional oligonucleotides comprises DNA, RNA, or a combination thereof.

22. The SNA of claim 20 or claim 21, wherein the one or more additional oligonucleotides comprises single-stranded DNA, double-stranded DNA, single-stranded RNA, double-stranded RNA, or a combination thereof.

23. The SNA of any one of claims 20-22, wherein the one or more additional oligonucleotides is an immunostimulatory oligonucleotide, an inhibitory oligonucleotide, an oligonucleotide that inactivates signal transducer and activator of transcription 3 (STAT3), or a combination thereof.

24. The SNA of claim 23, wherein the inhibitory oligonucleotide is an antisense oligonucleotide, small interfering RNA (siRNA), an aptamer, a short hairpin RNA (shRNA), a DNAzyme, or an aptazyme.

25. The SNA of claim 23 or claim 24, wherein the immunostimulatory oligonucleotide is a CpG-motif containing oligonucleotide, a double-stranded DNA oligonucleotide, or a single-stranded RNA oligonucleotide.

26. The SNA of any one of claims 23-25, wherein the immunostimulatory oligonucleotide is a toll-like receptor (TLR) agonist.

27. The SNA of claim 26, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

28. The SNA of any one of claims 23-27, wherein the inhibitory oligonucleotide is an antisense oligonucleotide, small interfering RNA (siRNA), an aptamer, a short hairpin RNA (shRNA), a DNAzyme, or an aptazyme.

29. The SNA of any one of claims 1-28, further comprising an antigen.

30. The SNA of claim 29, wherein the antigen is attached to one or more oligonucleotides in the shell of oligonucleotides.

31. The SNA of claim 29 or claim 30, wherein the antigen is attached to the surface of the SNA.

32. The SNA of any one of claims 29-31, wherein the antigen is encapsulated in the nanoparticle core.

33. The SNA of any one of claims 29-32, wherein the antigen is a tumor associated antigen, a tumor specific antigen, a neo-antigen, or a combination thereof.

34. The SNA of claim 33, wherein the antigen is OVA1, MSLN, P53, Ras, mutant IDH1 (IDH1 R132H), a melanoma related antigen, a HPV related antigen, a prostate cancer related antigen, a glioblastoma antigen, a grade IV astrocytoma antigen, an ovarian cancer related antigen, a breast cancer related antigen, a hepatocellular carcinoma related antigen, a bowel cancer related antigen, or human papillomavirus (HPV) E7 nuclear protein.

35. The SNA of any one of claims 1-34, wherein the SNA is from about 1 to about 150 nanometers (nm) in diameter.

36. The SNA of any one of claims 1-35, wherein the shell of oligonucleotides comprises about 4 to about 250 oligonucleotides.

37. The SNA of any one of claims 1-36, wherein each oligonucleotide in the shell of oligonucleotides is about 15 to about 100 base pairs in length.

38. A composition comprising a plurality of the spherical nucleic acids (SNAs) of any one of claims 1-37.

39. A method of producing an immune response to cancer in a subject, comprising administering to the subject an effective amount of the spherical nucleic acid (SNA) of any one of claims 1-37, the composition of claim 38, or a combination thereof, thereby producing an immune response to cancer in the subject.

40. A method of treating and/or ameliorating a cancer in a subject comprising administering to the subject an effective amount of the SNA of any one of claims 1-37, the composition of claim 38, or a combination thereof.

41. The method of claim 39 or 40, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

42. The method of any one of claims 39-41, wherein the cancer is glioblastoma.

43. The method of any one of claims 39-42, wherein the administering is by oral administration, topical administration, intravenous administration, intraarterial administration, mucosal administration, intraperitoneal administration, intramuscular administration, intratumoral administration, parenteral administration, intradermal administration, intranasal administration, subcutaneous administration, or a combination thereof.

44. The method of any one of claims 39-42, wherein the administering is by direct intracranial/intratumoral administration.

45. The method of any one of claims 39-44, wherein the subject is female.

46. The method of claim 45, wherein the administering is by intranasal administration.

47. The method of any one of claims 39-46, wherein the administering is combined with focused ultrasound (FUS).


SEQUENCE LISTING (10 sequences)

SEQ ID NO: 1 - DNA, 15 bp, Synthetic construct
  source 1..15: mol_type = other DNA
catttcccgtaaatc

SEQ ID NO: 2 - DNA, 15 bp, Synthetic construct
  source 1..15: mol_type = other DNA
gatttacgggaaatg

SEQ ID NO: 3 - DNA, 45 bp, Synthetic construct
  source 1..45: mol_type = other DNA
acatctagtacatgtctagtcagtatctagtgatcatctagacat

SEQ ID NO: 4 - DNA, 45 bp, Synthetic construct
  source 1..45: mol_type = other DNA
atgtctagatgatcactagatactgactagacatgtactagatgt

SEQ ID NO: 5 - DNA, 45 bp, Synthetic construct
  source 1..45: mol_type = other DNA
tacagatctactagtgatctatgactgatctgtacatgatctaca

SEQ ID NO: 6 - DNA, 43 bp, Synthetic construct
  source 1..43: mol_type = other DNA
acatctagtacatgtctagtcagtatctagtgatcatcagaca

SEQ ID NO: 7 - DNA, 15 bp, Synthetic construct
  source 1..15: mol_type = other DNA
ctaaatgccctttac

SEQ ID NO: 8 - DNA, 15 bp, Synthetic construct
  source 1..15: mol_type = other DNA
gtaaagggcatttag

SEQ ID NO: 9 - DNA, 45 bp, Synthetic construct
  source 1..45: mol_type = other DNA
tacaacatttcccgtaaatcggggggatttacgggaaatgttgta

SEQ ID NO: 10 - DNA, 45 bp, Synthetic construct; identical to SEQ ID NO: 5
  source 1..45: mol_type = other DNA